(19) United States
(12) Patent Application Publication
(10) Pub. No.: US 2026/0334860 A1
(43) Pub. Date: Oct. 29, 2026

(54) METHODS FOR SLEEP MONITORING AND INTERVENTION

(71) Applicant: Axilab, Inc., San Jose, CA (US)

(72) Inventors: Yinjuan HE, San Jose, CA (US); Zhe YU, San Jose, CA (US)

(21) Appl. No.: 19/656,264

(22) Filed: Apr. 23, 2026

Related U.S. Application Data
(60) Provisional application No. 63/793,190, filed on Apr. 23, 2025.

(51) Int. Cl. A61B 5/00; A61B 5/11; A61M 21/00; A61M 21/02

(52) U.S. Cl. CPC A61B 5/4809; A61B 5/1126; A61B 5/4818; A61B 5/725; A61B 5/7271; A61M 21/02; A61B 2560/0242; A61B 2562/0204; A61B 2562/0219; A61B 2562/0271; A61B 2562/029; A61M 2021/0022; A61M 2205/3303; A61M 2205/3306; A61M 2205/3317; A61M 2205/3368; A61M 2205/3375; A61M 2205/3592; A61M 2205/50; A61M 2230/005; A61M 2230/04; A61M 2230/40; A61M 2230/62; A61M 2230/63

(57) ABSTRACT

Embodiments of the present disclosure are directed toward a sleep monitoring and intervention system and can include, in some embodiments, a wearable device with PPG and IMU sensors for physiological monitoring and a vibration motor for neurostimulation delivery. In some embodiments, the system performs multidimensional anti-spoofing wear detection using cascaded decision rules and asymmetric smoothing feature extraction with fast-attack and slow-decay characteristics. In some embodiments, an awake prediction model inputs smoothed features into a modified Oakley Arousal Scoring Model to predict sleep states and trigger adaptive interventions, a bedside hub monitors environmental factors including ambient light, acoustic events, temperature, and humidity, and/or a cloud-based process reconstructs physiological feature tensors using deep learning networks with cascaded layers for comprehensive sleep staging classification and metric quantification.

### RELATED APPLICATIONS

This application claims benefit of and priority to provisional patent application no. 63/793,190 filed on Apr. 23, 2025 entitled “Wearable System for Bio-Signal Monitoring and Closed-Loop Neuromodulation,” the entire contents of which are incorporated herein by reference.

### FIELD OF THE DISCLOSURE

The present disclosure relates to wearable health technologies, specifically to a closed-loop control system for sleep monitoring and intelligent sleep aid based on an end-cloud collaborative architecture.

### BACKGROUND

Traditional sleep monitoring devices may lack sophisticated real-time processing capabilities and environmental integration for effective closed-loop intervention. Existing systems can operate with significant delays between detection and intervention, which may limit their effectiveness in preventing sleep disruptions. Furthermore, conventional approaches may fail to integrate comprehensive environmental monitoring with physiological sensing, potentially missing factors that can influence sleep quality.

Current sleep aid devices may rely on predetermined intervention patterns rather than adaptive, real-time responses to physiological changes. This can result in interventions that may be poorly timed or inappropriate for the user's current sleep state, potentially causing more disruption than benefit.

Accordingly, there exists a need for an integrated sleep monitoring system, and preferably, such a system further include intervention, which can process complex bio signals both at the edge of sleep-wake states for immediate response.

### SUMMARY

Embodiments of the disclosure address the needs of the state of the art for aiding individuals to maintain sleep and provide an integrated sleep monitoring system, and in some embodiments, an integrated sleep monitoring system which includes a cloud-based component for processing information and providing decisions for facilitating sleep for a user of a wearable device upon the user possibly waking up.

In general, embodiments of the present disclosure relates to systems and methods for monitoring, analyzing, and influencing sleep states of a user through coordinated operation of distributed computing components. In some embodiments, the system may include a plurality of interconnected elements, such as a wearable device configured to acquire physiological and/or motion-related signals, a bedside hub configured to aggregate and locally process data, one or more remote servers configured to perform advanced analytics and model execution, and optionally one or more external user devices (e.g., smartphones, tablets, or personal computers) configured to provide user interaction, visualization, and control (for example). The system architecture is modular, and functionality described herein may be performed by any one or more of these components individually or in combination.

In some embodiments, one or more components of the system execute software instructions to perform continuous or intermittent monitoring of sleep-related parameters. Such parameters may include, without limitation, heart rate, heart rate variability, respiration, body movement, environmental conditions, and other biometric or contextual data. The collected data may be processed locally, remotely, or in a hybrid manner to determine sleep stages, detect disturbances, identify patterns over time, and generate predictive or real-time assessments of sleep quality or user state. Processing may involve rule-based logic, statistical analysis, machine learning models, or combinations thereof, and may be dynamically updated or personalized based on user-specific data.

The system may further be configured to implement one or more intervention methodologies in response to detected or predicted sleep conditions. Interventions may include, for example, auditory, visual, tactile, or environmental adjustments delivered, in some embodiments, via one or more system components (in some embodiments, external components such as HVAC, window shades, and the like). In some cases, a wearable device may provide neuromodulation, while the bedside hub (for example), or connected environment control (for example, may control lighting, soundscapes, temperature, or other ambient factors. The timing, type, and intensity of interventions may be determined based on current or predicted sleep stages, historical user response patterns, or predefined protocols, and may be optimized through adaptive or learning-based techniques.

In some implementations, the external device may serve as an interface for user configuration, feedback, and reporting. Users may input preferences, review sleep metrics, and adjust system behavior through software applications executed on such devices. Additionally, the system may support bidirectional communication among components, enabling synchronization of data, distributed computation, and coordinated actuation of interventions. Security, privacy, and data integrity measures may be incorporated at various levels of the system, including encryption, anonymization, and access control. In some embodiments, the bedside hub may be considered the external device.

The disclosed methods may include steps performed by one or more of the system components, either sequentially or concurrently, and may be implemented in hardware, software, firmware, or any combination thereof. It should be understood that the allocation of functionality across components is not fixed; tasks described as being performed by one component may, in alternative embodiments, be performed by another component or distributed across multiple components. This flexibility allows the system to accommodate varying hardware capabilities, connectivity conditions, and deployment environments while maintaining consistent functional outcomes.

Although specific examples of system configurations and operations are described, these are intended to be illustrative rather than limiting. The scope of the disclosure encompasses variations in component composition, data sources, processing techniques, and intervention modalities, including implementations in which some components are omitted, combined, or substituted. The described approach enables scalable, adaptable, and user-centric sleep monitoring and intervention across a wide range of use cases and technical ecosystems.

Accordingly, some embodiments of the disclosure provide a comprehensive sleep monitoring and intervention system that may include both a wearable component and a bedside environmental hub. The system can track bio signals including heart rate, heart rate variability, respiratory rate, and movement patterns. The bedside dock may simultaneously monitor the external sleep environment by tracking ambient light, noise, temperature, and humidity.

The system, in some embodiments, may fuse wearable biometric data with environmental data to create a complete digitalized profile of the user's sleep architecture and surroundings. Based on this data, the system can provide real-time, adaptive neurostimulation to optimize health outcomes through a closed-loop control.

The wearable component (which may be referred to as a device, such terms used interchangeably throughout the present disclosure), or the system as a whole, may operate automatically without manual input and can include capability to interface with external smart home devices to enhance interventions through coordinated environmental modifications.

In some embodiments, a wearable neuromodulation system is provided and includes at least one bio-signal sensor (e.g., generating data associated with physiological information from a user), which is configured to detect one or more physiological parameters of a user, at least one processor configured to analyze the bio-signals and determine the user's physiological or cognitive state, an actuator/vibration module configured to deliver real-time (and in some embodiments, closed-loop) vibrations to the user (i.e., neuromodulation) via the wearable portion of the system, based on the determined state of the user.

In some embodiments, a bio-signal sensor can by any one or more of a photoplethysmography (PPG) sensor, an electrocardiography (ECG) sensor, an electroencephalography (EEG) sensor, respiratory rate sensor, a skin temperature sensor, a galvanic skin response (GSR) sensor, and an electromyography (EMG) sensor.

Embodiments of the disclosure utilize one or more processors, either local or remote (e.g., remote server(s), to provide, inter alia, machine learning inference for real-time classification of the user's state.

The actuator, which in addition to or in place of providing neuromodulation via vibrations applied to the user (e.g., skin of the wrist, arm, leg, chest, abdomen) can be structure which is configured to deliver one or more of: electrical pulses, thermal output, ultrasonic stimulation, or light-based cues. To this end, in some embodiments, the system can be configured to control one or more smart home devices to augment neuromodulation using environmental changes.

In some embodiments, methods are provided that perform real-time adaptive neuromodulation, by acquiring one or more bio-signals from a user via a wearable device, classifying the user's physiological or psychological state based on the bio-signals using an on-device inference model, and triggering a stimulation response tailored to said state through an integrated actuator. Classification can be supervised, unsupervised, or reinforcement learning models trained on multi-individual datasets. Moreover, stimulation response can be (as indicated throughout the disclosure), dynamically modulated vibration patterns synchronized to heart rate, breathing rhythm, or brainwave activity. In addition, in some embodiments,, and synchronization with smart home infrastructure is performed to adapt lighting, sound, or ambient temperature based on the user's detected state.

In some embodiments, a wearable device for a user is used to enhance sleep quality by identifying sleep stages and applying low-frequency stimulation that may or may not correspond to the user's bio-signal frequency as determined by methodology disclosed herein, so as to promote deeper sleep. For example, the system can be configured to be used to reduce stress by detecting heart rate variability and initiating calming interventions through feedback stimuli. Moreover, in some embodiments, the system can be used to improve focus by monitoring cognitive load and delivering stimulating feedback to maintain attention.

In some embodiments, a sleep monitoring and intervention system is provided and includes a wearable device comprising: a heart sensor to detect at least one of heart rate signals and blood volume pulses; an inertial measurement unit (IMU) sensor configured to detect at least one of micro-movements, gross movements, and postural position, a vibration motor configured to provide stimulation to at least one portion of the body with one or more stimulus patterns, and at least one processor including instructions operating thereon configured to cause the at least one processor to detect wake-arousal, and as a result thereof, cause the vibration motor to provide the stimulation so as to facilitate the user back to a sleeping state.

Such embodiments may include one and/or another of the following features, functions, functionality, structure, steps or clarifications (and if not mutually exclusive, in some embodiments, a plurality of, in some embodiments, a majority of, in some embodiments, substantially all of, and in some embodiments, all of), establishing even more embodiments according to the present disclosure:
the instructions are further configured to cause the processor to perform arousal scoring to detect wake-arousal;
the at least one processor comprises a plurality of processors;
a bedside hub configured to communicate with the wearable device;
a bedside hub includes at least one of: an ambient light sensor proximate an area of sleep of the user to monitor at least one of blue light exposure and light pollution, a microphone proximate the area of sleep to detect acoustic events including at least one of snoring, sleep apnea, and noise, and at least one of temperature and humidity sensors to monitor climate conditions proximate the area of sleep;
a/the bedside hub is configured to communicate with the wearable device;
the wearable device includes wireless communication means to communicate with an external device;
a/the bedside hub includes wireless communication means to communicate with the wearable device and/or an external device;
a/the external device includes a smartphone or other device configured for communication with a network;
a/the network comprises the internet, the cloud, LAN, and/or WAN;
at least one server;
a/the server comprises at least one processor with instructions operating thereon configured to cause the server to perform at least one of: a multimodal feature reconstruction using at least one of 1D convolutional kernels and Non-Maximum Suppression, and bio-analysis; multimodal feature reconstruction and/or bio-analysis is performed using deep learning networks;
use of deep-learning networks is with cascaded layers;
the heart sensor comprises a photoplethysmography (PPG) sensor;
the IMU comprises at least one accelerometer and/or at least one gyroscope;
the IMU comprises one or more 3-axis accelerometers and one or more gyroscopes, and wherein the one or more 3-axis accelerometers and/or the one or more gyroscopes detect the micro-movements, the gross movements, and/or the postural position;
the stimulus patterns are rhythmic;
a/the instructions are further configured to cause the at least one processor to detect use/wear of the wearable device;
detecting use/wear of the wearable includes anti-spoofing wear detection;
anti-spoofing wear detection uses cascaded decision rules addressing at least one of: signal saturation, severe light leakage, wearer stillness, edge-side physical activity feature extraction;
edge-side physical activity feature extraction includes asymmetric smoothing extraction; stimulation comprises neuromodulation;
stimulation comprises closed-loop neuromodulation;
arousal scoring occurs in real-time;
anti-spoofing wear detection comprises at least one of: collecting high-frequency at least one of PPG and IMU signals, down-sampling the at least one of PPG and IMU signals to a predetermined amount, using sliding windows, extracting at least one of: a signal mean, a signal standard deviation, and one or more inter-window mean differences, and applying cascaded decision rules to intercept invalid data from non-wearing states, activating subsequent calculation(s) only when a valid wearing condition detected;
asymmetric smoothing feature extraction performs frequency reduction and/or differential summation on high-frequency IMU signals;
asymmetric smoothing includes fast-attack and slow-decay characteristics to fill stationary gaps so as to capture signs corresponding to wake-arousal with limited to no delay;
asymmetric smoothing results in feature values to increase when body movement increases and decays slowly according to a preset forgetting factor when movement weakens, so as to effectively capture micro-movements in real-time;
fragmented micro-movements are captured in real-time;
closed-loop neuromodulation comprises an awake prediction model that inputs asymmetric smoothed features into a modified Oakley Arousal Scoring Model coupled with a weighted sliding window so as to predict light sleep or awake states significantly earlier than traditional algorithms;
closed-loop neuromodulation includes: evaluating continuous wake-up and high-frequency density trigger conditions based on wake-arousal scores; dynamically calculating vibration duration based on current wake-arousal score without waiting for commands, and/or higher arousal scores correspond to deeper wakefulness states resulting in longer neurostimulation periods;
the wearable device (and/or a/the bedside hub, an external device, a server) is configured to apply one or more filters to process data;
one or more filters detect one or more peaks and/or one or more valleys in time, and/or compress high-frequency raw waveform data into low-volume sequences of peak/valley timestamps;
low-volume sequences of peak/valley timestamps are communicated to a/the server;
the wearable device includes one or more filters configured to at least one of: apply a second-order cascaded Direct Form II Transposed high-pass and low-pass filter to PPG signals, detect peaks and valleys in time domain, and compress high-frequency raw waveform data into low-volume sequences of peak/valley timestamps for transmission to a/the server;
instructions operating on the at least one processor of the server (and/or the wearable device, the bedside hub, an external device) causes the processor to at least one of: parse received peak/valley sequences, calculate and merge time-sliding peak-to-peak interval distribution matrices, apply a one-dimensional convolutional kernel dual smoothing in distance and time domains, and apply Non-Maximum Suppression and dynamic weight penalties based on feature distance intervals to reconstruct noise-resistant, high-dimensional physiological feature tensors;
sleep staging classification model comprises a deep learning network including sequentially cascaded layers of: deep residual feature extraction, a Temporal Convolutional Network (TCN), a bidirectional LSTM, and a multi-head attention mechanism, so as to output continuous sleep stage labels comprising at least one of: awake, light sleep, deep sleep, and REM states;
instructions operating on the at least one processor of the server cause the server to additionally include performing physiological metric quantification;
physiological metric quantification comprises resting heart rate extraction and heart rate variability extraction;
resting heart rate extraction comprises slicing filtered feature vectors using specific duration sliding windows sorted by standard deviation to extract stable intervals and selecting windows with largest average pulse intervals;
heart rate variability extraction comprises calculating RMSSD in fixed windows after excluding pre-sleep and post-wake periods, while retaining intermediate intra-sleep awakenings, after removing outliers and excluding a predetermined percentage of anomalous data;
the predetermined percentage is: between: 5-90%, 10-90%, 15-90%, 20-90%, 30-90%, 40-90%, 50-90%, 60-90%, 70-90%, 80-90%, or ranges therebetween, less than approximately: 90%, 80%, 70%, 60%, 50%, 40%, 30%, 20%, 10%, 5%, or less than 5% or approximately 5%, 10%, 20%, 30%, 40%, 50%, 60%, 70%, 80% or 90%;
data from the PPG sensor is used by the system to analyze heart rate variability;
analysis of heart rate variability assesses autonomic nervous system balance and/or derives respiration rate from respiratory sinus arrhythmia during breathing cycles;
a/the IMU sensor is configured to differentiate between micro-movements and/or to determine sleep position;
a/the determined sleep position include any one or more of tossing and/or turning versus gross movements including one or more of waking up or getting out of bed;
a/the determined sleep position includes any one or more of back, side, and stomach positioning;
closed-loop neuromodulation includes an internal cool-down period and/or a rolling limit mechanism;
inclusion of the internal cool-down period and/or the rolling limit mechanism is configured to form a safe, instantaneous, and adaptive edge-side intervention loop;
instructions operating on the at least one processor of the wearable device and/or a/the server cause the at least one processor of the wearable device and/or the server to use linear frequency match logic to identify when a user is slipping out of sleep and applies rhythmic stimulus to anchor the user by matching heart rate frequency in a range of In some embodiments, the frequency range can be selected from 0.1-9 Hz, 0.2-9 Hz, 0.3-9 Hz, 0.4-8 Hz, 0.5-8 Hz, 0.5-7 Hz, 0.5-6 Hz, 0.5-5 Hz, 0.5-4 Hz, 0.5-3 Hz, 0.5-2 Hz, and ranges therebetween);
instructions operating on the at least one processor of the wearable device and/or the server cause the at least one processor of the wearable device and/or the server to limit one or more intervention zones to micro-awakenings, wakes, and light-sleep states, and/or to establish veto zones so as to never intervene during an REM or deep sleep state of a user;
the wearable device further comprises mm Wave radar for non-contact sensing;
instructions operating on one and/or another of one or more processors associated with the system include: HRV/heart rate mapping; Respiratory mapping; or HRV/heart rate mapping and respiratory mapping;
as well as any of the additional features, functions, functionality, structure, steps or clarifications, if not described immediately above (and if not mutually exclusive, in some embodiments, a plurality of, in some embodiments, a majority of, in some embodiments, substantially all of, and in some embodiments, all of), set out for earlier discussed embodiments (above), or elsewhere described herein (e.g., below).

In some embodiments, a method for sleep monitoring and intervention is provided and includes detecting at least one of heart rate signals and blood volume pulses of a user/individual (e.g., using a heart sensor of a wearable device) and/or detecting at least one of micro-movements, gross movements, and/or postural position of the user; detecting or determining wake-arousal of the user; and as a result of detecting wake-arousal, applying or causing application of stimulation to at least one portion of the body of the user with one or more stimulus patterns so as to facilitate the user back to a sleeping state.

Such embodiments may include one and/or another of the following features, functions, functionality, structure, steps or clarifications (and if not mutually exclusive, in some embodiments, a plurality of, in some embodiments, a majority of, in some embodiments, substantially all of, and in some embodiments, all of), establishing even more embodiments according to the present disclosure:
performing arousal scoring to detect wake-arousal of the user;
the steps are performed via at least one of a wearable device, a bedside hub, and an external device and/or a server;
communicating with a/the bedside hub;
monitoring at least one of blue light exposure proximate an area of sleep of the user and light pollution using an ambient light sensor, detecting proximate the area of sleep acoustic events of the user including at least one of snoring, sleep apnea, and noise using a microphone; and monitoring climate conditions proximate the area of sleep using at least one of temperature and humidity sensors;
communicating between the bedside hub and the wearable device;
communicating with an external device using wireless communication means of the wearable device;
communicating between a/the wearable device and/or an external device using wireless communication means of the bedside hub;
the external device includes a smartphone or other device configured for communication with a network;
a/the network comprises the internet, the cloud, a LAN, and/or a WAN; using a/the server to perform processing and analysis operations, or, such processing and analysis operations are performed on the wearable device, an external device, or a bedside hub;
performing at least one of: a multimodal feature reconstruction using at least one of 1D convolutional kernels and Non-Maximum Suppression, and bio-analysis;
multimodal feature reconstruction and/or bio-analysis is performed using deep learning networks;
use of deep-learning networks is with cascaded layers;
detecting utilizes an IMU which comprises at least one accelerometer and/or at least one gyroscope;
detecting utilizes an IMU which comprises one or more 3-axis accelerometers and one or more gyroscopes, and wherein detecting at least one of micro-movements, gross movements, and postural position uses the one or more 3-axis accelerometers and/or the one or more gyroscopes;
the stimulus patterns are rhythmic;
detecting use/wear of the wearable device on the user;
detecting use/wear of the wearable includes anti-spoofing wear detection;
anti-spoofing wear detection uses cascaded decision rules addressing at least one of: signal saturation, severe light leakage, wearer stillness, edge-side physical activity feature extraction;
edge-side physical activity feature extraction includes asymmetric smoothing extraction;
stimulation comprises neuromodulation;
stimulation comprises closed-loop neuromodulation;
arousal scoring, which may occur in real-time or substantially in real-time;
anti-spoofing wear detection comprises at least one of: collecting high-frequency at least one of PPG and IMU signals of the user, down-sampling the at least one of PPG and IMU signals to a predetermined frequency, using sliding windows, extracting at least one of: a signal mean, a signal standard deviation, and one or more inter-window mean differences, and applying cascaded decision rules to intercept invalid data from non-wearing states, activating subsequent calculation(s) only when a valid wearing condition detected;
asymmetric smoothing feature extraction performs frequency reduction and/or differential summation on high-frequency IMU signals;
asymmetric smoothing includes fast-attack and slow-decay characteristics to fill stationary gaps so as to capture signs corresponding to wake-arousal with limited to no delay;
asymmetric smoothing results in feature values to increase when body movement increases and decays slowly according to a preset forgetting factor when movement weakens, so as to effectively capture micro-movements in real-time;
fragmented micro-movements of the user are captured in real-time;
closed-loop neuromodulation comprises using an awake prediction model that inputs asymmetric smoothed features into a modified Oakley Arousal Scoring Model coupled with a weighted sliding window so as to predict light sleep or awake states significantly earlier than traditional algorithms;
evaluating continuous wake-up and high-frequency density of the user trigger conditions based on wake-arousal scores of the user; and dynamically calculating vibration duration based on current wake-arousal score without waiting for commands, wherein higher arousal scores correspond to deeper wakefulness states resulting in longer neurostimulation periods;
filtering data using at least one of the wearable device, a/the server, a/the bedside hub, and an external device;
filtering to detect one or more peaks and/or one or more valleys in time, and/or compress high-frequency raw waveform data into low-volume sequences of peak/valley timestamps;
communicating the low-volume sequences of peak/valley timestamps to a/the server;
filtering data to apply a second-order cascaded Direct Form II Transposed high-pass and low-pass filter to PPG signals, detect peaks and valleys in time domain, and compress high-frequency raw waveform data into low-volume sequences of peak/valley timestamps for transmission to at least one of a/the bedside hub, an/the external device, and a/the server;
at least one of parsing received peak/valley sequences, calculating and merge time-sliding peak-to-peak interval distribution matrices, applying a one-dimensional convolutional kernel dual smoothing in distance and time domains, and applying Non-Maximum Suppression and dynamic weight penalties based on feature distance intervals to reconstruct noise-resistant, high-dimensional physiological feature tensors;
applying a sleep staging classification model including cascaded layers of deep residual feature extraction, Temporal Convolutional Network, bidirectional LSTM, and multi-head attention, so as to output continuous sleep stage labels comprising at least one of: awake, light sleep, deep sleep, and REM states;
performing physiological metric quantification;
physiological metric quantification comprises performing resting heart rate extraction and heart rate variability extraction;
performing resting heart rate extraction comprises slicing filtered feature vectors using specific duration sliding windows sorted by standard deviation to extract most stable intervals and selecting windows with largest average pulse intervals;
performing heart rate variability extraction comprises calculating RMSSD in fixed windows after excluding pre-sleep and post-wake periods, while retaining intermediate intra-sleep awakenings, after removing outliers and excluding a predetermined percentage of anomalous data;
a/the predetermined percentage is: between: 5-90%, 10-90%, 15-90%, 20-90%, 30-90%, 40-90%, 50-90%, 60-90%, 70-90%, 80-90%, or ranges therebetween, less than approximately: 90%, 80%, 70%, 60%, 50%, 40%, 30%, 20%, 10%, 5%, or less than 5% or approximately 5%, 10%, 20%, 30%, 40%, 50%, 60%, 70%, 80% or 90%;
using data from the PPG sensor to analyze heart rate variability;
analysis of heart rate variability to assess autonomic nervous system balance and/or deriving respiration rate from respiratory sinus arrhythmia during breathing cycles;
differentiating between micro-movements and/or to determine sleep position;
a/the determined sleep position include any one or more of tossing and/or turning versus gross movements including one or more of waking up or getting out of bed;
determined sleep position includes any one or more of back, side, and stomach positioning;
closed-loop neuromodulation includes using an internal cool-down period and/or a rolling limit mechanism;
inclusion of the internal cool-down period and/or the rolling limit mechanism forms a safe, instantaneous, and adaptive edge-side intervention loop;
using linear frequency match logic to identify when a user is slipping out of sleep and applying rhythmic stimulus to anchor the user by matching heart rate frequency in a predetermined frequency range (selected predetermined range, e.g., 0.1-9 Hz, 0.2-9 Hz, 0.3-9 Hz, 0.4-8 Hz, 0.5-8 Hz, 0.5-7 Hz, 0.5-6 Hz, 0.5-5 Hz, 0.5-4 Hz, 0.5-3 Hz, 0.5-2Hz, and ranges therebetween);
limiting one or more intervention zones to micro-awakenings, wakes, and light-sleep states, and/or establishing veto zones so as to never intervene during an REM or deep sleep state of a user;
performing non-contact sensing via mmWave radar;
HRV/heart rate mapping, Respiratory mapping, or HRV/heart rate mapping and respiratory mapping;
as well as any of the additional features, functions, functionality, structure, steps or clarifications, if not described immediately above (and if not mutually exclusive, in some embodiments, a plurality of, in some embodiments, a majority of, in some embodiments, substantially all of, and in some embodiments, all of), set out for earlier discussed embodiments (above), or elsewhere described herein (e.g., below).

In some embodiments, a sleep monitoring and intervention system is provided and includes a wearable device comprising a heart sensor to detect at least one of heart rate signals and blood volume pulses, an inertial measurement unit (IMU) sensor configured to detect at least one of micro-movements, gross movements, and postural position; a vibration motor configured to provide stimulation to at least one portion of the body with one or more stimulus patterns; and at least one processor including instructions operating thereon configured to cause the at least one processor to detect wake-arousal, and as a result thereof, cause the vibration motor to provide the stimulation so as to facilitate the user back to a sleeping state, a bedside hub configured to communicate with at least the wearable device and including at least one processor with instructions operating thereon; and a server and/or an external device comprising at least one processor with instructions operating thereon configured to cause the server to perform at least one of: a multimodal feature reconstruction using at least one of 1D convolutional kernels and Non-Maximum Suppression, and bio-analysis.

Such embodiments may include one and/or another of the following features, functions, functionality, structure, steps or clarifications (and if not mutually exclusive, in some embodiments, a plurality of, in some embodiments, a majority of, in some embodiments, substantially all of, and in some embodiments, all of), establishing even more embodiments according to the present disclosure:
the instructions operating on at least one of the processor of the wearable device, the bedside hub, and the server are configured to cause the processor to perform arousal scoring to detect wake-arousal;
the at least one processor of any of the wearable device, the bedside hub and the server comprises a plurality of processors;
a/the bedside hub includes at least one of: an ambient light sensor to monitor at least one of blue light exposure and light pollution of the user, a microphone to detect acoustic events of or near the user including at least one of snoring, sleep apnea, and noise, and at least one of temperature and humidity sensors to monitor climate conditions;
a/the external device includes a smartphone or other device configured for communication with a network;
as well as any of the additional features, functions, functionality, structure, steps or clarifications, if not described immediately above (and if not mutually exclusive, in some embodiments, a plurality of, in some embodiments, a majority of, in some embodiments, substantially all of, and in some embodiments, all of), set out for earlier discussed embodiments (above), or elsewhere described herein (e.g., below).

In some embodiments, a sleep monitoring and intervention wearable device is provided and includes a heart sensor to detect at least one of heart rate signals and blood volume pulses; an inertial measurement unit (IMU) sensor configured to detect at least one of micro-movements, gross movements, and postural position; a vibration motor configured to provide stimulation to at least one portion of the body with one or more stimulus patterns; and at least one processor including instructions operating thereon configured to cause the at least one processor to detect wake-arousal, and as a result thereof, cause the vibration motor to provide the stimulation so as to facilitate the user back to a sleeping state.

Such embodiments may include one and/or another of the following features, functions, functionality, structure, steps or clarifications (and if not mutually exclusive, in some embodiments, a plurality of, in some embodiments, a majority of, in some embodiments, substantially all of, and in some embodiments, all of), establishing even more embodiments according to the present disclosure:
the device communicates with at least one of: a bedside hub; and an external device and/or a remote server;
instructions are further configured to cause the at least one processor of the device to perform at least one of: a multimodal feature reconstruction using at least one of 1D convolutional kernels and Non-Maximum Suppression, and bio-analysis;
instructions are further configured to cause the at least one processor of at least one of the device, the bedside hub, the external device and the remote server to perform at least one of: a multimodal feature reconstruction using at least one of 1D convolutional kernels and Non-Maximum Suppression, and bio-analysis;
instructions operating on at least one of the processor of the wearable device (and/or bedside hub, and/or external device, and/or server) cause the processor to perform arousal scoring to detect wake-arousal;
instructions operating on at least one of the processor of the wearable device, the bedside hub, the external device, cause the at least one processor to perform arousal scoring to detect wake-arousal;
the wearable device includes at least one of: an ambient light sensor to monitor at least one of blue light exposure and light pollution, a microphone to detect acoustic events including at least one of snoring, sleep apnea, and noise; and at least one of temperature and humidity sensors to monitor climate conditions proximate an area of sleep;
at least one of the wearable device, the bedside hub, and the external device includes at least one of: an ambient light sensor to monitor at least one of blue light exposure and light pollution, a microphone to detect acoustic events including at least one of snoring, sleep apnea, and noise; and at least one of temperature and humidity sensors to monitor climate conditions proximate an area of sleep;
a/the external device includes a smartphone or other device configured for communication with a network;
as well as any of the additional features, functions, functionality, structure, steps or clarifications, if not described immediately above (and if not mutually exclusive, in some embodiments, a plurality of, in some embodiments, a majority of, in some embodiments, substantially all of, and in some embodiments, all of), set out for earlier discussed embodiments (above), or elsewhere described herein (e.g., below).

One of skill in the art will appreciate that while some embodiments of the disclosure have processes/steps performed on a network (e.g., cloud, one or more servers, and the like), in some embodiments, such processes, or some cases, portions of such processes (include deep-learning/AI related functionality) can be before on one or more of a wearable device, a bedside hub, an external device (e.g., smartphone, laptop/PC and the like).

These and other embodiments, features, structure, steps, functions, functionality, and clarifications, as well as objects and advantages thereof, will become even more evident with the following detailed description and drawings, a brief description of which is set out below.

### BRIEF DESCRIPTION OF THE DRAWINGS

A better understanding of the features and advantages of the present embodiments and features of inventive concepts disclosed herein may be obtained by reference to the following detailed description that sets forth illustrative embodiments, in which the principles of the inventive concepts are utilized, and the accompanying drawings of which:

FIG. 1 shows a diagram of an overview of a system for sleeping monitoring and intervention, according to some embodiments of the present disclosure.

FIG. 2 shows a detailed schematic of a wearable device for a system for sleep monitoring and intervention, according to some embodiments of the present disclosure.

FIG. 3 shows a perspective view (including a cutaway of internals thereof) of a bedside hub for a system for sleep monitoring and intervention, according to some embodiments of the present disclosure.

FIG. 4 shows a perspective view of a server for use with a system for sleep monitoring and intervention, according to some embodiments of the present disclosure.

FIG. 5 illustrates a system operation flowchart showing an overall process flow for a system for sleep monitoring and intervention, according to some embodiments of the present disclosure.

FIG. 6 illustrates a multi-layered architecture of a system for sleep monitoring and intervention, according to some embodiments of the present disclosure.

FIG. 7 illustrates a detailed edge-side processing flowchart showing a device-side process for use with a system for sleep monitoring and intervention, according to some embodiments of the present disclosure.

FIG. 8 illustrates a cloud-side processing flowchart for use with a system for sleep monitoring and intervention, according to some embodiments of the present disclosure.

### DETAILED DESCRIPTION

Embodiments of the present disclosure provides a sleep monitoring and intervention system that integrates wearable physiological sensing with environmental monitoring and, in some embodiments, cloud-based analytics to deliver adaptive neurostimulation interventions. The system combines real-time edge processing for immediate intervention responses with comprehensive cloud-based analysis for long-term sleep optimization. The architecture enables both instantaneous sleep disruption prevention and sophisticated longitudinal sleep pattern analysis through coordinated operation of multiple interconnected components that can work together to provide personalized sleep improvement strategies.

### System Overview

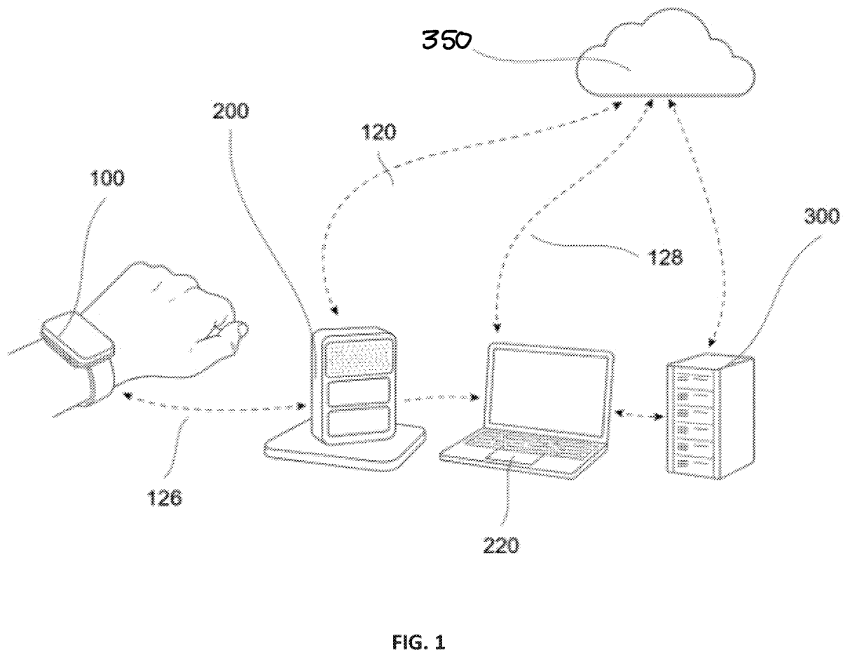

Referring to FIG. 1, a sleep monitoring and intervention system 10 (which in some embodiments, is a closed-loop system) may include a comprehensive architecture that integrates multiple components for real-time physiological monitoring and adaptive intervention. The system 10 can include a wearable device 100 that may be worn by a user during sleep periods, a bedside hub 200 positioned proximate to the user's sleep environment, a remote server 300 configured for advanced data processing, and cloud infrastructure 350 that can facilitate data storage and analysis. Communication pathways 120, 126, 128 can connect these components to enable seamless data flow and coordinated system operation.

In some embodiments, the wearable device 100 may continuously monitor physiological parameters and provide immediate intervention capabilities through on-board processing and stimulation mechanisms. The bedside hub 200 is configured, in some embodiments, to simultaneously monitor environmental conditions that may influence sleep quality, while the remote server 300 (via cloud infrastructure 350) can perform analysis of collected data to optimize long-term sleep patterns and system performance.

In some embodiments, the system architecture can enable both immediate edge-based responses for time-critical interventions and comprehensive cloud-based analysis for long-term optimization. This dual-processing approach, in some embodiments, ensures that sleep disruptions are quicky addressed with little to no delay while maintaining (in some embodiments) detailed historical analysis for personalized sleep improvement strategies.

The wearable device 100 shown in FIG. 1 may establish wireless communication with the bedside hub 200 through low-power protocols that can include Bluetooth Low Energy or proprietary wireless communication standards. The bedside hub 200 can also maintain internet connectivity through Wi-Fi or ethernet connections to facilitate data transmission to the remote server 300. The cloud infrastructure 350 provides scalable computing resources for advanced machine learning operations (for detecting sleep interruptions, for example) and long-term data storage capabilities. Bi-directional communication pathways 120, 126, 128, in some embodiments, can enable real-time coordination between edge processing and cloud analytics, allowing the system 10 to balance immediate response requirements with comprehensive analysis capabilities. One of skill in the art will appreciate the “real-time” is events occurring at the same time, or nearly at the same time, with a limited delay (e.g., within seconds, in some embodiments, and within tenths of seconds or less, in some embodiments). Accordingly, references to “real-time” correspond to at the same time or within such limited timing.

Local PC 220 may be included in system 10, in some embodiments, and can include intermediate processing capabilities positioned between the bedside hub 200 and remote server 300 to provide local data management and processing coordination. The local PC 220 can comprise standard computing hardware that may include multi-core processors, sufficient memory for data buffering and processing, and storage systems for temporary data retention and system configuration management.

In some embodiments, local PC 220 may serve as a data aggregation point that can collect information from multiple wearable devices 100 and bedside hubs 200 within a household or facility. The processing capabilities can include preliminary data analysis, quality assessment, and data formatting for efficient transmission to the remote server 300. Local processing may reduce bandwidth requirements and improve system responsiveness by handling routine data management tasks without requiring cloud connectivity.

In some embodiments, the PC, modified with additional environmental sensing/sensors, can take the place of the bedside hub.

### Wearable Device

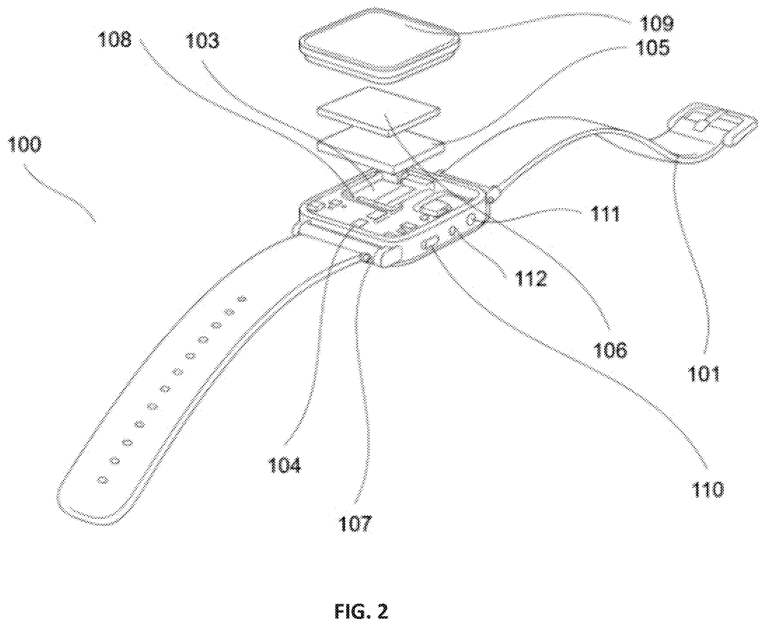

Referring to FIG. 2, the wearable device 100 may include a comprehensive hardware architecture designed for continuous physiological monitoring and precise neurostimulation delivery. The user mounting means 101 can comprise an adjustable wrist strap or band system that may be constructed from biocompatible materials configured to maintain secure positioning during sleep while ensuring user comfort throughout extended wear periods. The mounting system 101 may include quick-release mechanisms and adjustable sizing to accommodate various wrist dimensions and user preferences.

In some embodiments, a processor 103 within the wearable device 100 may comprise a low-power microcontroller or system-on-chip architecture that can execute real-time signal processing algorithms while maintaining minimal power consumption. The processor 103 can include dedicated hardware accelerators for digital signal processing operations and may support concurrent execution of multiple processing threads for simultaneous sensor data acquisition and analysis. Integrated memory systems can provide both volatile storage for active processing operations and non-volatile storage for firmware, calibration parameters, and temporary data buffering.

In some embodiments, a communication module 104 may be included for enabling one or more, and preferably, multi-protocol wireless connectivity that can include Bluetooth for local device pairing, Wi-Fi for direct internet access, and cellular communication capabilities for remote operation scenarios. The communication module 104 may implement adaptive power management that can adjust transmission power and frequency based on signal strength requirements and battery status. Security protocols can ensure encrypted data transmission and authenticated device pairing to protect user physiological information.

In some embodiments, the wearable device 100 may include a battery system 105 comprising, for example, high-energy-density lithium-ion or lithium-polymer cells configured to provide multi-day operation between charging cycles. Intelligent power management circuitry can also be included to monitor battery status, optimize charging cycles, and implement dynamic power scaling based on usage patterns and processing demands. The battery system 105 may include safety protection circuits that can prevent overcharging, over-discharge, and thermal runaway conditions.

The wearable device 100 can also include a vibration motor/actuator 106 (such phrases/terms used interchangeably) which may be a Linear Resonant Actuator (LRA) or similar precision actuator capable of generating controlled vibration frequencies and amplitudes across a wide dynamic range. The actuator 106 can be configured to deliver haptic feedback patterns that may include variable frequency, amplitude, and duration characteristics tailored to specific intervention requirements to maintain sleep. Drive circuitry may provide precise control over stimulation parameters and can implement safety limits to prevent excessive stimulation intensity.

In some embodiments, the wearable device further includes a PPG sensor 107, which may comprise multiple photodiodes and LED emitters configured to detect blood volume changes through optical measurement techniques. The sensor 107 can include multiple wavelengths of light emission that may enable enhanced signal quality and artifact rejection. Analog front-end circuitry may provide signal conditioning, amplification, and analog-to-digital conversion with high resolution and low noise characteristics. The sensor positioning within the device 100 can ensure consistent skin contact while minimizing motion artifacts during sleep movements.

In some embodiments, the wearable device 100 can include an IMU sensor 108 which can include a multi-axis accelerometers (in some embodiments, one or more) and gyroscopes (in some embodiments, one or more) that can detect any and all of (and preferably all of) micro-movements, gross movements, and postural positioning-preferably with high sensitivity and wide dynamic range. The sensor 108 can provide, in some embodiments, three-dimensional motion detection capabilities that can differentiation between subtle sleep-related movements and significant position changes. Digital signal processing within the IMU 108 (or via processor 103) can include motion filtering, calibration compensation, and real-time feature extraction to support immediate movement analysis.

In some embodiments, the wearable device 100 can include an optional display 109, which may be a low-power display technology that can include OLED, e-ink, or similar technologies configured to provide user feedback (or the user acquire information) while minimizing power consumption and light emission during sleep periods. The display 109 can present system status information, sleep quality metrics, and user interaction prompts through an intuitive interface design. Brightness control and sleep mode functionality may ensure minimal circadian disruption while maintaining essential user communication capabilities.

In some embodiments, the wearable device 100 can include one or more physical input buttons 110 which may include tactile switches configured for easy operation in low-light conditions typical of sleep environments. The button configuration 110 can include an on/activate switch 111 for power control and system activation, along with additional input buttons that may enable user interaction with device functions, settings adjustment, and manual intervention control. Button design may incorporate tactile feedback and ergonomic positioning to facilitate intuitive operation without visual confirmation.

### Bedside Hub

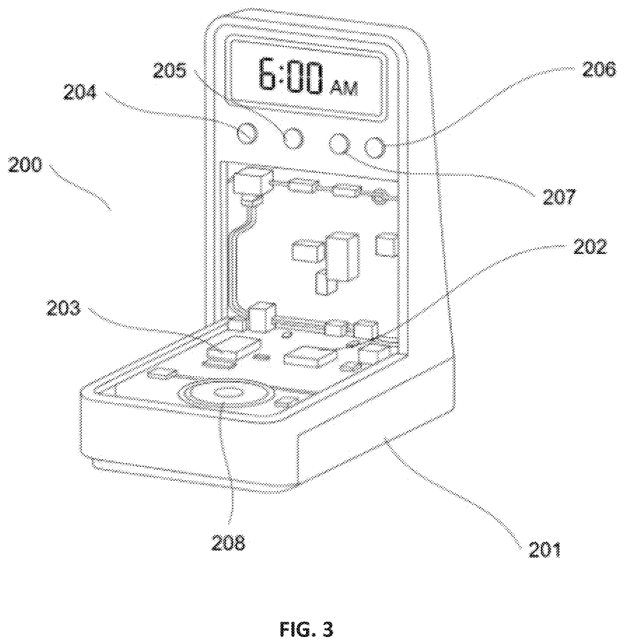

Referring to FIG. 3, the bedside hub 200 may include a comprehensive environmental monitoring and device support system configured to assess external factors that can influence sleep quality while providing charging and connectivity services for the wearable device (for example). A power supply system 201 of the hub can include both battery backup capability for uninterrupted operation during power outages and external power connection through standard AC adapters or USB-C power delivery systems (power cord not shown). Power management circuitry can be included to optimize energy consumption across multiple sensor systems and communication modules while maintaining continuous environmental monitoring capabilities.

In some embodiments, a processor 202 within the bedside hub 200 can include a processing unit (which may be more powerful than the wearable device processor 103 but can also use a similar processor as in the wearable device), which can coordinate sensor data collection, local environmental analysis, and multi-device communication management. The processor 202 can include sufficient computational resources to perform real-time environmental data processing, pattern recognition for acoustic events, and coordination of smart home device integration. Memory provided in the hub 200 can provide both active processing storage and historical data retention for environmental trend analysis.

In some embodiments, the hub 200 includes a communication module 203 to provide simultaneous multi-protocol connectivity, which can include Bluetooth for wearable device communication, Wi-Fi for internet connectivity and smart home integration, and additional protocols such as Zigbee or Z-Wave for comprehensive smart home device compatibility. The communication system 203 may also be used to support mesh networking capabilities that may enable coordination with multiple wearable devices and integration with existing home automation systems.

In some embodiments, the hub 200 can include an ambient light sensor 204 which may comprise photodiodes configured to detect light intensity across multiple wavelengths that can include blue light spectrum monitoring for circadian rhythm assessment. The sensor 204 can provide continuous monitoring of light pollution levels during sleep periods and may detect artificial light sources that could disrupt natural sleep cycles. Included calibration capabilities can ensure accurate light measurement across varying environmental conditions and seasonal changes.

A microphone 205 may be included, in some embodiments, and can be a high-sensitivity acoustic sensor for detecting and analyzing sleep-related audio events while maintaining user privacy through local processing. The microphone 205 can be configured to capture snoring patterns, potential sleep apnea indicators, and external noise disturbances that may include traffic, neighbors, or household sounds. Digital signal processing capabilities may enable real-time audio analysis and event classification without requiring audio data transmission to external servers.

A temperature sensor 206 and a humidity sensor 207, provided in the hub 200, can include precision environmental monitoring components configured to measure sleep micro-climate conditions with high accuracy and stability. The sensors 206, 207 thus can provide continuous monitoring of thermal comfort parameters that may significantly impact the user's ability to reach and maintain deep sleep states. Sensor positioning within the hub 200 can ensure representative measurement of the sleep environment while avoiding interference from the device's own heat generation.

In some embodiments, wireless charging capability 208 of the hub can be an inductive charging system configured to provide convenient charging of the wearable device 100 without requiring physical connection. The charging system 208 can include foreign object detection, thermal management, and efficiency optimization to ensure safe and reliable charging operations. Charging status indication and automatic charging termination may prevent overcharging while maintaining device readiness for continuous use.

In some embodiments, a user interface 209 may be included with the hub and include visual displays, indicator lights, and/or audio feedback systems so as to provide environmental status information and system alerts while minimizing sleep disruption. The interface 209 can include adjustable brightness levels and/or sleep mode functionality to reduce light emission during designated sleep periods while maintaining essential status communication capabilities.

### Server Architecture

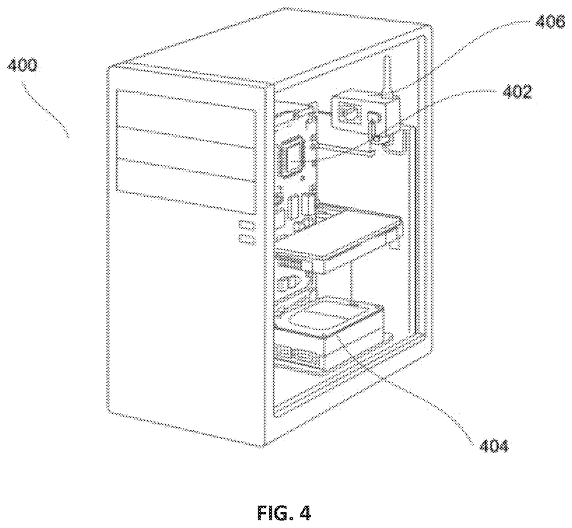

FIG. 4 illustrates a server architecture 400 which may include a computing infrastructure specifically configured for physiological data analysis and machine learning operations according to some embodiments. To this end, a processor subsystem 402 can comprise multiple processing cores that may include general-purpose CPUs for system management and specialized AI/ML accelerators such as GPUs or TPUs for deep learning computations. High-speed memory systems may provide both system RAM for active processing operations and high-bandwidth memory for machine learning model execution.

In some embodiments, a data storage system 404 for the server may include tiered storage architecture that can include high-speed solid-state storage for active data processing, high-capacity storage for historical data retention, and archival storage systems for long-term data preservation. The storage system 405 can implement data redundancy and backup mechanisms to ensure data integrity and availability. Database management systems may optimize data organization and retrieval for both real-time analysis requirements and historical trend analysis.

In some embodiments, a communication module 406 of the server enables high-bandwidth internet connectivity through multiple connection types that can include fiber optic, broadband, and cellular backup connections. The communication system 406 can support secure data transmission protocols that may include end-to-end encryption, authentication mechanisms, and data integrity verification. Load balancing and redundancy capabilities may ensure reliable connectivity for critical system operations and continuous data synchronization with edge devices (e.g., environment synchronization).

In some embodiments, the server 400 may include distributed computing capabilities that can scale processing resources based on user load and computational demands. Container orchestration and microservices architecture may enable efficient resource utilization and system scalability. Monitoring and logging systems can provide comprehensive system health assessment and performance optimization capabilities.

### General System Operation Flow

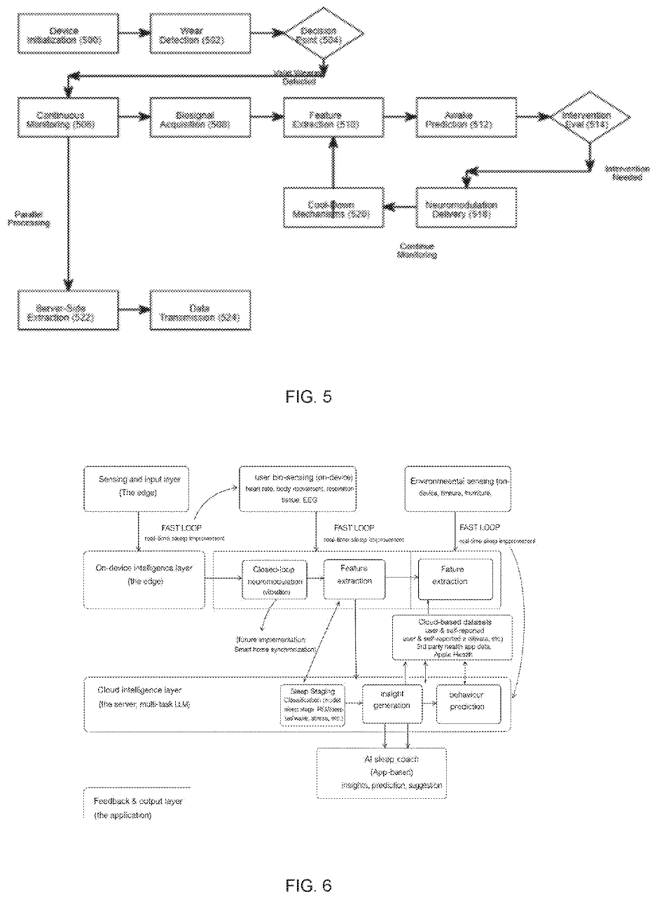

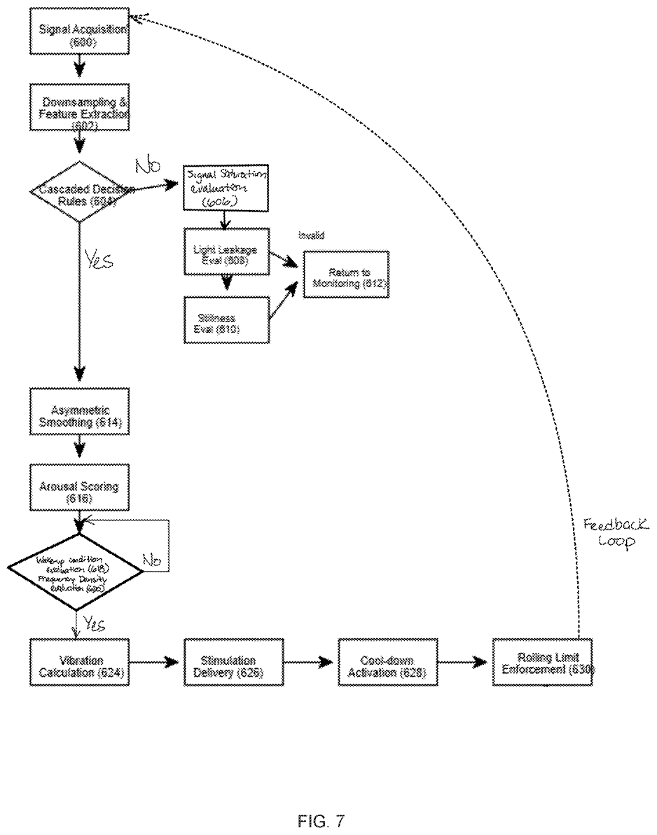

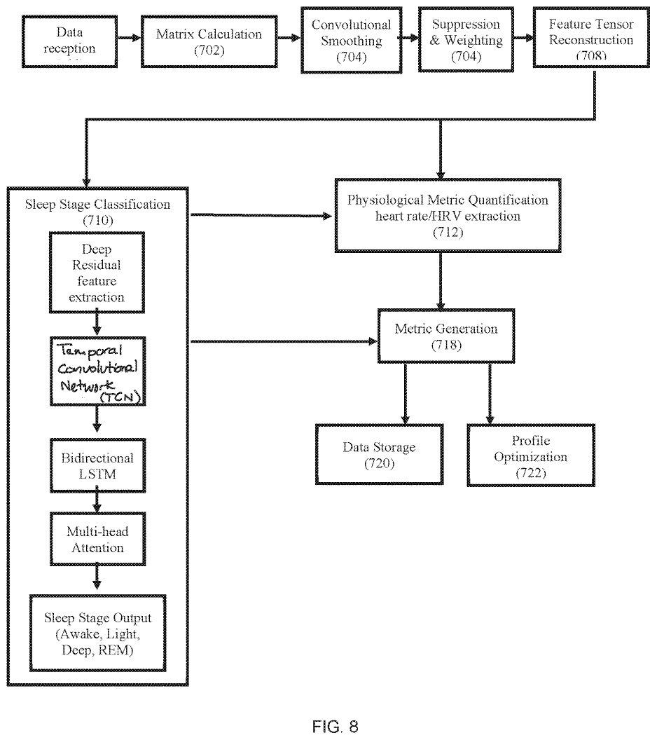

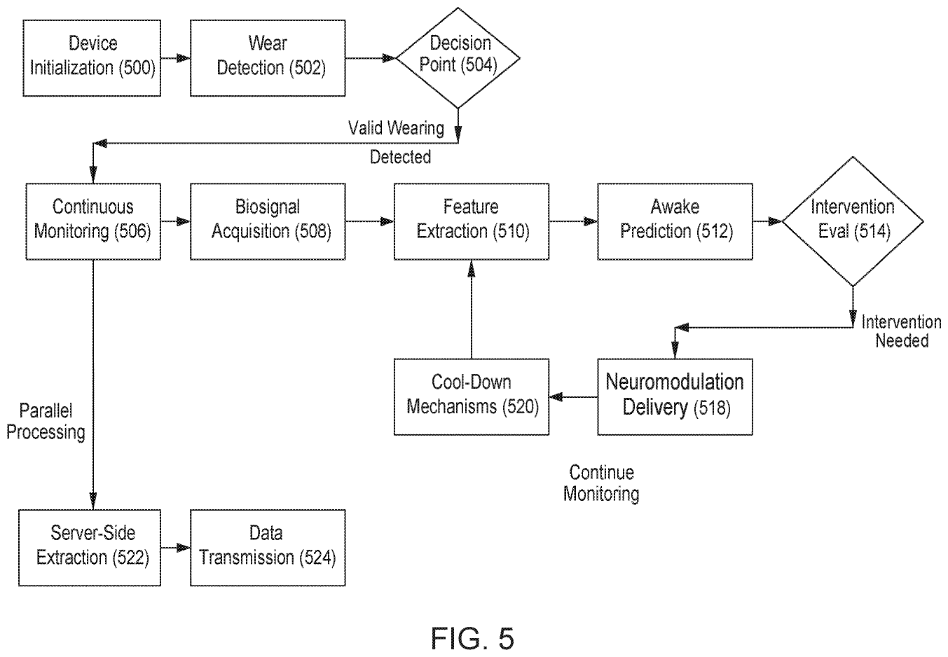

FIG. 5 illustrates a system operation flowchart, according to some embodiments, illustrating a process flow for sleep monitoring and intervention that begins with device initialization procedures 500, according to some embodiments. The initialization process 500 can include sensor calibration routines that may establish baseline parameters for PPG and IMU sensors, communication establishment between wearable device 100 and bedside hub 200, and system configuration loading that can include user-specific parameters and intervention preferences.

In some embodiments, wear detection processes 502 are used and implement multidimensional validation to verify proper device positioning and sensor contact quality before activating monitoring functions. The detection system 502 can be used to evaluate PPG signal quality, IMU baseline readings, and skin contact impedance to ensure reliable physiological data acquisition. Anti-spoofing mechanisms may prevent false positive detection from environmental factors or improper device placement.

In some embodiments, a decision point 504 evaluates wear detection results through cascaded decision rules (or rule) that can assess signal saturation levels, light leakage conditions, and absolute stillness indicators. When valid wearing conditions are confirmed, the system can proceed to active monitoring mode 506, while invalid conditions may return the system to standby mode to conserve battery power and prevent false data processing.

In some embodiments, monitoring mode 506 may be continuous, and includes, in some embodiments, simultaneous bio-signal acquisition 508 from one or more (and preferably, multiple) sensors that can capture various signals, including PPG signals for cardiovascular monitoring and/or IMU signals for movement detection. Sampling, in some embodiments, high-frequency sampling, can be performed with, in some embodiments, real-time buffering so as to ensure comprehensive data capture while managing memory and processing resources efficiently.

In some embodiments, a feature extraction process(es) 510 are provided to analyze collected bio-signals through algorithms that can identify patterns indicative of sleep state changes. In some embodiments, extraction 510 implements asymmetric smoothing techniques with fast-attack and slow-decay characteristics to capture subtle movement patterns that often precede awakening events. Signal processing can include frequency domain analysis, time-domain feature calculation, and pattern recognition algorithms optimized for real-time execution.

In some embodiments, awake prediction algorithms 512 process extracted features through, for example, modified Oakley Arousal Scoring Models that can incorporate weighted sliding windows and asymmetric prior features. The prediction process 512 can, in some embodiments, generate real-time wake-arousal scores that can indicate the likelihood of sleep disruption or awakening events significantly earlier than traditional monitoring approaches. Predictive capabilities may enable proactive intervention before full awakening occurs.

In some embodiments, intervention evaluation 514 may assess detected arousal patterns against predetermined criteria that can include continuous wake-up conditions and high-frequency density thresholds. The evaluation 514 can consider current sleep stage, intervention history, and user-specific response patterns to determine appropriate intervention strategies. Safety mechanisms may prevent intervention during protected sleep stages such as REM or deep sleep periods.

When intervention criteria are satisfied, delivery of neuromodulation 518 can be executed (via the vibration motor of the wearable device) through controlled vibration patterns that can be dynamically calculated based on current wake-arousal scores. Higher wake-arousal scores, in some embodiments, may correspond to deeper wakefulness states and can result in longer stimulation periods with adjusted intensity levels. The delivery 518 can be implemented via precise timing control and pattern generation to optimize intervention effectiveness while maintaining user comfort.

In some embodiments, cool-down 520 may be activated following intervention delivery to prevent excessive stimulation and ensure safe operation intervals. Cool-down 520 can be implemented both during fixed timing periods, as well as adaptive intervals based on user response patterns and intervention effectiveness. Rolling limit enforcement can be used to track cumulative intervention frequency and duration to maintain safe stimulation levels throughout the sleep period.

In some embodiments, parallel to edge-based processing/server-side feature extraction 522 can be used to compress physiological data for cloud transmission. This process 522 can use second-order cascaded filtering to detect peaks and valleys in PPG signals and compress high-frequency waveform data into low-volume timestamp sequences. Periodic data transmission 524 can be used to maintain synchronization between edge and cloud components while minimizing power consumption and bandwidth requirements.

### Multi-Layered System

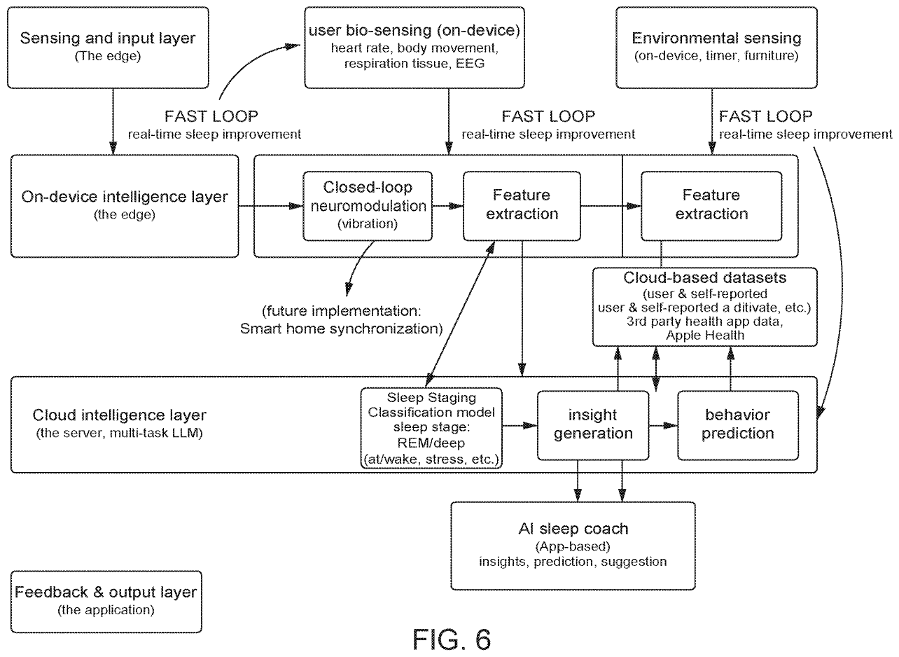

Similar to FIG. 5, FIG. 6 is another system operation flow, according to some embodiments, which presents a multi-layered sleep monitoring and improvement system with four main operational layers that operate together to provide real-time sleep optimization and, in some embodiments, can provide long-term behavioral improvement. Accordingly, the sensing and input layer can include user bio-sensing capabilities with on-device monitoring of, for example, one or more (and preferably a plurality, and in some embodiments, all of) heart rate, body movement, respiration, tissue characteristics, ECG and/or EEG signals, along with environmental sensing capabilities that may monitor, for example, one or more of (and preferably a plurality, and in some embodiments, all of) on-device conditions, temperature, and humidity for comprehensive sleep environment assessment.

In some embodiments, the on-device intelligence layer may include closed-loop neuromodulation through vibration delivery, feature extraction processes, and additional feature extraction capabilities that can utilize cloud-based datasets including user and self-reported data, third party health application data, and Apple Health integration. This layer can enable immediate response capabilities while maintaining connection to broader health data ecosystems for enhanced personalization and accuracy.

In some embodiments, a cloud intelligence layer can be provided and can include a multi-task large language model (server-based, in some embodiments) that can perform sleep staging classification for sleep stages including any or more of a plurality of REM, deep, awake, focus, cognitive and stress states, along with, in some embodiments, insight generation and behavior prediction capabilities. Cloud processing/intelligence layer can leverage computing resources to analyze physiological patterns and generate personalized recommendations based on comprehensive data analysis.

In some embodiments, a feedback and output layer may include an AI sleep coach application that can provide insights, predictions, and suggestions to users through intuitive interfaces and personalized guidance systems. As illustrated, fast loop connections all for real-time (or near real-time, both terms/phrases used interchangeably) sleep improvement between the sensing layer and on-device intelligence layer, as well as feedback loop connections for optimization that can connect the cloud intelligence layer back to the feature extraction processes for continuous system improvement.

### Edge-Side & Server/Cloud Side Processing

While certain processes, in some embodiments, are carried out on the wearable device, the hub, and the cloud/server, in some embodiments, all processes could be carried out on the edge-side (wearable device and/or hub), although machine learning and artificial intelligence aspects (sleep staging, sleep scoring, insight generation, behavior prediction, and an AI sleep coach aspect, due to the processing power required, are carried out on a remote server(s)/cloud.

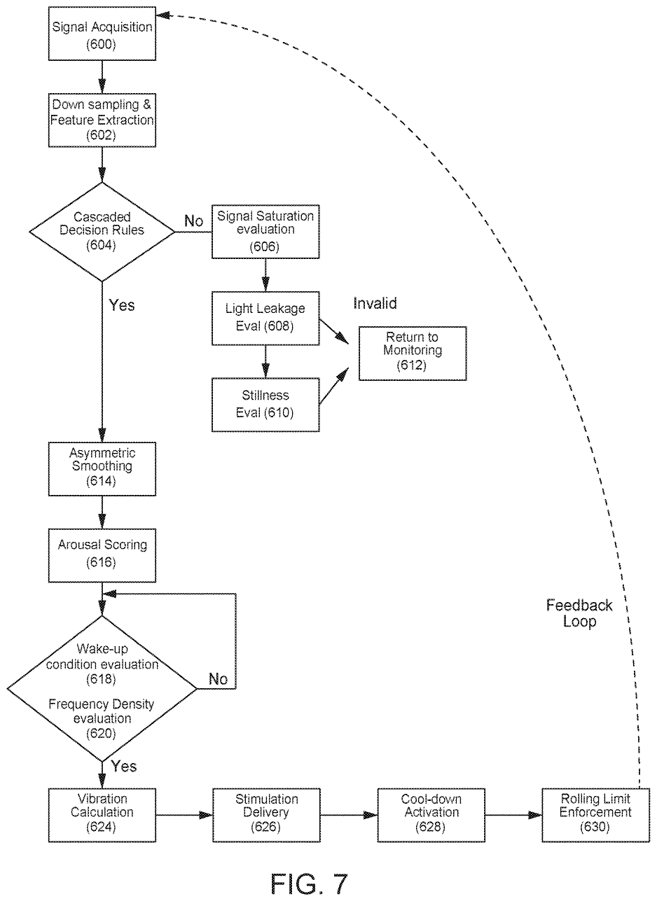

Accordingly, as shown in FIG. 7, which substantially corresponds to an edge-side processing flow, a signal processing pipeline executed, in some embodiments, on the wearable device 100 for immediate response capabilities. Signal acquisition 600 can simultaneously collect data from multiple sensors at appropriate sampling rates that may include PPG signals at frequencies sufficient to capture cardiovascular dynamics and IMU signals at rates capable of detecting subtle movement patterns. Acquisition 600 can implement real-time buffering and preliminary filtering to manage data flow (to the hub and/or server) and reduce processing overhead.

In some embodiments, down-sampling and feature extraction processes 602 may reduce data volume while preserving critical physiological information through intelligent sampling techniques. The system can apply anti-aliasing filters before down-sampling to prevent signal distortion and may implement adaptive sampling rates based on detected signal characteristics. Feature extraction algorithms can be used to calculate statistical measures that may include signal means, standard deviations, and inter-window differences to characterize physiological patterns.

Cascaded decision rules 604 can be used, in some embodiments, to implement multi-stage validation of signal quality and device wearing status through systematic evaluation of multiple signal characteristics. Signal saturation evaluation 606 can be used to detect when sensor readings exceed valid measurement ranges due to excessive ambient light, sensor malfunction, or improper positioning. Saturation evaluation 606 can implement threshold-based detection with hysteresis to prevent oscillation between valid and invalid states.

In some embodiments, light leakage evaluation 608 identifies conditions where optical sensors receive ambient light rather than physiological signals from skin contact. The evaluation 608 can analyze signal characteristics that may include DC offset levels, AC component amplitude, and frequency content to distinguish between valid physiological signals and ambient light contamination. Adaptive thresholds may account for varying environmental lighting conditions and skin tone variations.

Stillness evaluation 610 can be used to distinguish between natural sleep stillness and device removal or improper positioning through analysis of IMU signal characteristics. The evaluation 610 therefore can assess movement patterns, gravitational orientation, and/or signal stability to determine device wearing status. Time-based analysis may prevent false detection from brief periods of natural stillness during deep sleep phases.

In some embodiments, when invalid conditions are detected through any stage of the cascaded decision rules 604, the system can be configured to return to monitoring mode 612 without proceeding to intervention calculations, thereby conserving processing resources and preventing false interventions. Valid signals can proceed to asymmetric smoothing processes 614 that apply fast-attack and slow-decay characteristics specifically designed to capture transient movement patterns that precede awakening events.

In some embodiments, asymmetric smoothing 614 may implement frequency reduction and/or differential summation on high-frequency IMU signals (for collected data from the wearable device and/or the hub) through mathematical operations that emphasize movement onset while maintaining sensitivity to subtle changes. Fast-attack characteristic can be used to cause feature values to rise instantly when body movement increases, while slow-decay characteristics can maintain elevated values according to preset forgetting factors when movement weakens. This approach may effectively fill stationary gaps in movement data to capture early arousal signs without delay.

In some embodiments, on the cloud/server side (but in some embodiments can be performed on the wearable device, the hub, and/or local PC), arousal scoring 616 can be used to process smoothed features through modified Oakley Arousal Scoring Models (for example) that incorporate weighted sliding windows and asymmetric prior features. Scoring 616 can generate real-time assessments of sleep state transitions and can be used to predict light sleep or awake states significantly earlier than traditional monitoring approaches. Scoring can implement adaptive thresholds based on individual user patterns and historical response data.

In some embodiments, wake-up condition evaluation 618 and frequency density evaluation 620 may assess whether detected arousal patterns meet predetermined intervention criteria through analysis of both instantaneous arousal scores and temporal patterns. Evaluations 618, 620 can use continuous wake-up indicators which suggest sustained arousal states and high-frequency density patterns that can indicate rapid sleep state fluctuations. Multi-criteria assessment can also be used to ensure intervention appropriateness while preventing unnecessary stimulation.

In some embodiments, when intervention criteria are satisfied, vibration calculation 624 can be used to determine appropriate stimulation parameters based on current arousal scores and user-specific response patterns. Calculation 624 can implement dynamic parameter mapping that can be used to adjust vibration duration, intensity, and/or frequency based on detected arousal depth. Higher arousal scores often correspond to deeper wakefulness states and can result in longer intervention periods with modified stimulation characteristics.

The delivery of stimulation 626, in some embodiments, execute calculated intervention patterns via control of the vibration motor 106 with real-time parameter adjustment capabilities (according to some embodiments). Stimulation delivery 626 implements pattern generation that may include variable frequency, amplitude modulation, and/or temporal sequencing preferably optimized for sleep state guidance. Feedback monitoring during stimulation can be used to enable real-time adjustment of intervention parameters based on physiological response.

In some embodiments, cool-down 628 and rolling limit enforcement 630 can be used to ensure safe operation intervals and prevent excessive stimulation through systematic tracking of intervention frequency and cumulative exposure. Cool-down 628 can be implemented both in fixed minimum intervals between interventions and adaptive periods based on user response patterns and intervention effectiveness. Rolling limit enforcement 630 can be used to track cumulative stimulation over extended periods to maintain safe exposure levels and prevent habituation effects.

FIG. 8 illustrates generally a process flow with respect to cloud-side processing, according to some embodiments, which helps to illustrate analytical capabilities executed on server infrastructure 400 for comprehensive sleep analysis and long-term optimization. Accordingly, compressed data can be collected 700 including physiological data transmitted from edge devices through secure communication channels that can utilize encryption, authentication, and/or data integrity verification. Reception of data 700 can be configured to handle multiple concurrent data streams from numerous devices while maintaining real-time processing capabilities.

In some embodiments, matrix calculation 702 can be used to process received peak/valley sequences to generate time-sliding peak-to-peak interval distribution matrices. Calculation 702 can utilize parallel processing techniques to handle large datasets efficiently and may apply statistical analysis methods to identify physiological patterns and anomalies. Matrix calculation can include correlation analysis, spectral analysis, and/or temporal pattern recognition optimized for cardiovascular signal processing.

In some embodiments, convolutional smoothing 704 can be used and apply one-dimensional convolutional kernels to perform dual smoothing operations in distance and/or time domains. Smoothing 704 can include adaptive kernel parameters that perform adjusting based on signal characteristics and/or noise levels. Multi-scale analysis capabilities can capture both fine-grained physiological details and broader temporal patterns that may indicate sleep architecture changes.

Suppression and weighting 706 can be used, in some embodiments, to implement Non-Maximum Suppression algorithms and/or dynamic weight penalties based on feature distance intervals to enhance signal quality and reduce artifacts. Such suppression and weighting 706 can be used to identify and eliminate redundant or conflicting signal components while preserving physiologically relevant information. Dynamic weighting may adjust feature importance based on signal quality metrics and temporal consistency patterns.

In some embodiments, feature tensor reconstruction 708 can be used to combine processed data streams from multiple sources to create comprehensive, noise-resistant, high-dimensional physiological feature representations. Reconstruction 708 implements tensor algebra operations to preserve spatial and temporal relationships within physiological data while reducing dimensionality for efficient processing. Multi-modal fusion techniques can integrate cardiovascular, movement, and environmental data into unified feature representations.

In some embodiments, a reconstructed feature tensor from block 708 is input into the first layer of the sleep staging classification model 710, where it is processed sequentially through the cascaded deep residual, TCN, LSTM, and attention layers to generate the final sleep stage output.

In some embodiments, the sleep staging classification model 710 is a deep learning network with sequentially cascaded internal layers: deep residual feature extraction, a Temporal Convolutional Network (TCN), a bidirectional LSTM, and a multi-head attention mechanism.

Sleep stage classification 710, using deep learning networks can use cascaded layers specifically designed for physiological signal analysis. Accordingly, deep residual feature extraction layers can be used to capture non-linear relationships within physiological data while preventing gradient vanishing in deep network architectures. Temporal Convolutional Network (TCN) components can be used to model long-term temporal dependencies that may be critical for accurate sleep stage transitions.

In some embodiments, Bidirectional LSTM layers can be used to capture forward and/or backward temporal relationships within sleep data to improve classification accuracy for ambiguous sleep state transitions. Multi-head attention mechanisms can identify and weight the most relevant features for sleep stage determination while adapting to individual user patterns. Classification 710 can output continuous sleep stage labels that may include Awake, Light Sleep, Deep Sleep, and REM states with confidence scores and transition probabilities.

In some embodiments, physiological metric quantification 712 can be used to extract physiological parameters for detailed health assessment and sleep quality evaluation including heart rate extraction 714 and heart rate variability (HRV) extraction 716. Heart rate extraction 714, in some embodiments, identifies stable measurement periods through statistical analysis of heart rate variability and can implement outlier detection to ensure accurate resting heart rate calculation. Extraction 714 can be used in some embodiments to apply sliding window techniques with standard deviation sorting to identify the most stable intervals for baseline measurements.

In some embodiments, HRV extraction 716 can be used to calculate Root Mean Square of Successive Differences (RMSSD) after excluding pre-sleep and post-wake periods, while retaining intermediate intra-sleep awakenings, to assess autonomic nervous system function during sleep. Extraction 716, in some embodiments, implements fixed window analysis with outlier removal from both ends of measurement windows and can be configured to exclude predetermined percentages of anomalous data to derive robust nightly HRV measurements. Statistical validation techniques can also be used to ensure measurement reliability and clinical relevance.

In some embodiments, metric generation 718 receives sleep stage labels from the sleep staging classification model 710 to compile comprehensive sleep quality assessments, including sleep stage distribution analysis and arousal frequency quantification. Such metric generation 718 can be used to create personalized sleep reports with trend analysis and additionally, in some embodiments, identify patterns that correlate with sleep quality improvements or degradation. Comparative analysis capabilities can also be used to benchmark individual performance against population norms and historical personal data.

Data storage 720 can be used, in some embodiments, to maintain comprehensive historical records for longitudinal analysis and machine learning model training. Such storage can implement data lifecycle management that may include active data retention, archival processes, and/or data anonymization for research applications. Security measures can also be used to ensure user privacy protection while enabling valuable population-level sleep research.

In some embodiments, profile optimization 722 refines personalized intervention strategies from accumulated data, and additionally, improve system effectiveness over time through machine learning techniques. To this end, optimization 722 can be used to identify individual response patterns to different intervention strategies and, in some embodiments, can adapt stimulation parameters, timing, and/or frequency based on measured effectiveness. Continuous learning capabilities can also improve system performance through user-specific model refinement and population-level pattern recognition.

In some embodiments, edge-side physical activity feature extraction and adaptive low-latency sleep aid trigger (see FIGS. 6 and 7) is used to capture fragmented micro-movements in real-time at the edge to trigger zero or near zero-latency adaptive intervention. Such processing can include three distinct sub-components that work together to provide immediate response capabilities without cloud dependency.

In some embodiments, asymmetric smoothing feature extraction 614 performs frequency reduction and/or differential summation on high-frequency IMU signals, which introduces an asymmetric smoothing strategy with fast-attack and slow decay characteristics. As such, feature values can rise instantly when body movement increases and decay slowly according to a preset forgetting factor when movement weakens. Such an approach can effectively fill stationary gaps to capture early signs of arousal without delay, which may enable the system 10 to detect subtle changes in sleep state that precede full awakening.

In some embodiments, an awake prediction model 512 (FIG. 5) can be used to input the asymmetric smoothed features into a modified Oakley Arousal Scoring Model. By coupling asymmetric prior features with a weighted sliding window, the arousal score can jump rapidly upon initial contact with micro-movement signals, which may predict “light sleep/easy to wake” or “awake” states significantly earlier than traditional algorithms. This predictive capability can enable proactive intervention before the user fully awakens.

The closed-loop neuromodulation component, according to some embodiments, evaluate “continuous wake-up” and “high-frequency density” trigger conditions 618, 620 based on real-time arousal scores. Once trigger conditions are met, system 10 can dynamically calculate and map the duration of sleep-aid vibrations 624 based on the current arousal score without waiting for cloud commands. Higher arousal scores may indicate deeper states of wakefulness, which can result in longer vibration interventions. The system 10 may drive the motor 106 and combine an internal cool-down period 628 and rolling limit mechanism 630 to form a safe, instantaneous, and adaptive edge-side intervention loop.

In some embodiments, feature extraction 522 for server-side models may include applying a second-order cascaded Direct Form II Transposed high-pass and low-pass filter to the PPG signal Accordingly, the system 10 can detect peaks and valleys in the time domain, which may compress high-frequency raw waveform data into a low-volume sequence of peak/valley timestamps. This compression can reduce data transmission requirements while preserving physiological information for cloud-based analysis.

Transmitting data, in some embodiments, includes periodically reporting extracted valid peak/valley sequences and body movement features to server 400. This selective data transmission approach minimizes power consumption while ensuring that comprehensive physiological data reaches the cloud for advanced processing.

### Bio-Mirroring Intervention Strategy

In some embodiments, the system can use Linear Frequency Match logic to identify when a user is slipping out of sleep and apply rhythmic stimulus to anchor them, which can match their heart rate frequency in the range of 0.5-2 Hz (e.g., 0.1-9 Hz, 0.2-9 Hz, 0.3-9 Hz, 0.4-8 Hz, 0.5-8 Hz, 0.5-7 Hz, 0.5-6 Hz, 0.5-5 Hz, 0.5-4 Hz, 0.5-3 Hz, 0.5-2 Hz, and ranges therebetween). Intervention zones may be limited to micro-awakenings, wakes, and/or light sleep states. The system 10 can incorporate veto zones and may be programmed to never intervene during REM or deep sleep states to protect cognitive and physical recovery processes. Accordingly, in some embodiments, current frequency logic may match the user's instantaneous heart rate frequency in such ranges using the wearable PPG sensor 107 as the primary input.

### Adaptive Entrainment Capabilities

In some embodiments, respiratory mapping may be provided that shifts toward lower frequencies to match breathing patterns more closely tied to the parasympathetic nervous system. To this end, HRV-informed tuning can use heart rate variability as a sensitivity parameter, where low HRV indicating high stress may require slower, more stabilizing frequencies for neurostimulation, while high HRV indicating relaxed states can require lighter, more subtle stimulus.

In some embodiments, stage-specific mapping can be used to vary frequency for neurostimulation based on the depth of light sleep, which can use different frequencies for N1 versus N2 sleep stages to more effectively guide users back into deeper sleep states.

mmWave Radar Integration

In some embodiments, the system includes integration of millimeter-wave radar for non-contact sensing capabilities. Such mm-wave radar can be used to detect sub-millimeter chest wall movements to provide accurate respiration and heart rate data without requiring the user to wear a device. In some embodiments, such functionality can act as a redundant check for wearable sensor data and, moreover, can enable monitoring of a partner in the same bed, which can expand the system's monitoring capabilities beyond the primary user.

### Machine Learning/AI

In some embodiments, and as noted above, server-side machine learning and artificial intelligence capabilities can be used to provide predictive modeling that continuously refines the system's ability to anticipate sleep and wake-up patterns through comprehensive analysis of accumulated physiological and environmental data. Such AI systems can implement ensemble learning approaches that may combine multiple predictive models to achieve enhanced accuracy in sleep state prediction, with each model contributing specialized analysis of different physiological parameters and temporal patterns.

Accordingly, in some embodiments, machine learning may utilize deep neural network architectures specifically optimized for time-series physiological data analysis, which can include recurrent neural networks for temporal pattern recognition and convolutional neural networks for feature extraction from multi-dimensional sensor data. The AI models can implement transfer learning techniques that may leverage population-level sleep patterns to initialize personalized models, then adapt through continuous learning from individual user data to achieve increasingly accurate predictions tailored to specific physiological responses and sleep behaviors.

In some embodiments, predictive modeling may incorporate reinforcement learning algorithms that can optimize intervention strategies based on measured effectiveness of previous interventions, creating a feedback loop that continuously improves the system's ability to prevent sleep disruptions. In some embodiments, AI can analyze correlations between environmental conditions monitored by the bedside hub 200, physiological patterns detected by the wearable device 100, and intervention outcomes to identify optimal timing and stimulation parameters for individual users. Advanced feature engineering techniques can be used to extract physiological signatures that precede sleep state transitions, enabling the system to predict awakening events with increasing temporal precision as more data becomes available.

In some embodiments, cloud-based AI can implement federated learning approaches that can improve predictive accuracy across the user population while maintaining individual privacy through differential privacy techniques and secure aggregation protocols. The system can utilize unsupervised learning methods to identify novel sleep pattern clusters and anomalies that may indicate emerging health conditions or changes in sleep quality, providing early warning capabilities that extend beyond immediate intervention to long-term health monitoring. Adaptive model architectures may automatically adjust network complexity and feature selection based on data availability and prediction accuracy requirements, ensuring optimal performance across diverse user populations and varying data quality conditions.

### Exemplary Enumerated Embodiments

Embodiment 1. A sleep monitoring and intervention system comprising a wearable device comprising: a heart sensor to detect at least one of heart rate signals and blood volume pulses; an inertial measurement unit (IMU) sensor configured to detect at least one of micro-movements, gross movements, and postural position, a vibration motor configured to provide stimulation to at least one portion of the body with one or more stimulus patterns, and at least one processor including instructions operating thereon configured to cause the at least one processor to detect wake-arousal, and as a result thereof, cause the vibration motor to provide the stimulation so as to facilitate the user back to a sleeping state.

Embodiment 2. The system of embodiment 1, wherein the instructions are further configured to cause the processor to perform arousal scoring to detect wake-arousal.

Embodiment 3. The system of embodiments 1 or 2, wherein the at least one processor comprises a plurality of processors.

Embodiment 4. The system of any of embodiments 1-3, further comprising a bedside hub configured to communicate with the wearable device.

Embodiment 5. The system of any of embodiments 1-3, further comprising a bedside hub includes at least one of: an ambient light sensor to monitor at least one of blue light exposure and light pollution, a microphone to detect acoustic events including at least one of snoring, sleep apnea, and noise, and at least one of temperature and humidity sensors to monitor climate conditions proximate an area of sleep.

Embodiment 6. The system of embodiments 4 or 5, wherein the bedside hub is configured to communicate with the wearable device.

Embodiment 7. The system of any of embodiments 1-6, wherein the wearable device includes wireless communication means to communicate with an external device.

Embodiment 8. The system of any of embodiments 4-6, wherein the bedside hub includes wireless communication means to communicate with the wearable device and/or an external device.

Embodiment 9. The system of embodiments 7 or 8, wherein the external device includes a smartphone or other device configured for communication with a network.

Embodiment 10. The system of embodiment 9, wherein the network comprises the internet, the cloud, LAN, and/or WAN.

Embodiment 11. The system of any of embodiments 1-10, further comprising a server.

Embodiment 12. The system of embodiment 11, wherein the server comprises at least one processor with instructions operating thereon configured to cause the server to perform at least one of: a multimodal feature reconstruction using at least one of 1D convolutional kernels and Non-Maximum Suppression, and bio-analysis.

Embodiment 13. The system of embodiment 12, wherein multimodal feature reconstruction and/or bio-analysis is performed using deep learning networks.

Embodiment 14. The system of embodiment 13, wherein the use of deep-learning networks is with cascaded layers.

Embodiment 15. The system of any of embodiments 1-14, wherein the heart sensor comprises a photoplethysmography (PPG) sensor.

Embodiment 16. The system of any of embodiments 1-15, wherein the IMU comprises at least one accelerometer and/or at least one gyroscope.

Embodiment 17. The system of any of embodiments 1-15, wherein the IMU comprises one or more 3-axis accelerometers and one or more gyroscopes, and wherein the one or more 3-axis accelerometers and/or the one or more gyroscopes detect the micro-movements, the gross movements, and/or the postural position.

Embodiment 18. The system of any of embodiments 1-17, wherein the stimulus patterns are rhythmic.

Embodiment 19. The system of any of embodiments 1-18, wherein the instructions are further configured to cause the at least one processor to detect use/wear of the wearable device.

Embodiment 20. The system of embodiment 19, wherein the detecting use/wear of the wearable includes anti-spoofing wear detection.

Embodiment 21. The system of embodiment 20, wherein the anti-spoofing wear detection uses cascaded decision rules addressing at least one of: signal saturation, severe light leakage, wearer stillness, edge-side physical activity feature extraction.

Embodiment 22. The system of embodiment 21, wherein edge-side physical activity feature extraction includes asymmetric smoothing extraction.

Embodiment 23. The system of any of embodiment 1-22, wherein stimulation comprises neuromodulation.

Embodiment 24. The system of any of embodiments 1-22, wherein stimulation comprises closed-loop neuromodulation.

Embodiment 25. The system of any of embodiments 2-24, wherein arousal scoring occurs in real-time.

Embodiment 26. The system of any of embodiments 20-25, wherein anti-spoofing wear detection comprises at least one of: collecting high-frequency at least one of PPG and IMU signals, down-sampling the at least one of PPG and IMU signals to a predetermined amount, using sliding windows, extracting at least one of: a signal mean, a signal standard deviation, and one or more inter-window mean differences, and applying cascaded decision rules to intercept invalid data from non-wearing states, activating subsequent calculation(s) only when a valid wearing condition detected.

Embodiment 27. The system of any of embodiments 22-26, wherein asymmetric smoothing feature extraction performs frequency reduction and/or differential summation on high-frequency IMU signals.

Embodiment 28. The system of any of embodiments 22-27, wherein asymmetric smoothing includes fast-attack and slow-decay characteristics to fill stationary gaps so as to capture signs corresponding to wake-arousal with limited to no delay.

Embodiment 29. The system of any of embodiments 22-28, wherein asymmetric smoothing results in feature values to increase when body movement increases and decays slowly according to a preset forgetting factor when movement weakens, so as to effectively capture micro-in real-time.

Embodiment 30. The system of embodiment 29, wherein fragmented micro-movements are captured in real-time.

Embodiment 31. The system of any of embodiments 24-30, wherein closed-loop neuromodulation comprises an awake prediction model that inputs asymmetric smoothed features into a modified Oakley Arousal Scoring Model coupled with a weighted sliding window so as to predict light sleep or awake states significantly earlier than traditional algorithms.

Embodiment 32. The system of any of embodiments 24-31, wherein the closed-loop neuromodulation includes: evaluating continuous wake-up and high-frequency density trigger conditions based on wake-arousal scores; dynamically calculating vibration duration based on current wake-arousal score without waiting for commands, and/or higher arousal scores correspond to deeper wakefulness states resulting in longer neurostimulation periods.

Embodiment 33. The system of any of embodiments 1-32, wherein the wearable device is configured to apply one or more filters to process data.

Embodiment 34. The system of embodiment 33, wherein the one or more filters detect one or more peaks and/or one or more valleys in time, and/or compress high-frequency raw waveform data into low-volume sequences of peak/valley timestamps.

Embodiment 35. The system of embodiment 34, wherein the low-volume sequences of peak/valley timestamps are communicated to a/the server.

Embodiment 36. The system of any of embodiments 1-35, wherein the wearable device includes one or more filters configured to at least one of: apply a second-order cascaded Direct Form II Transposed high-pass and low-pass filter to PPG signals, detect peaks and valleys in time domain, and compress high-frequency raw waveform data into low-volume sequences of peak/valley timestamps for transmission to a/the server.

Embodiment 37. The system of any of embodiments 11-36, wherein instructions operating on the at least one processor of the server causes the server to at least one of: parse received peak/valley sequences, calculate and merge time-sliding peak-to-peak interval distribution matrices, apply a one-dimensional convolutional kernel dual smoothing in distance and time domains, and apply Non-Maximum Suppression and dynamic weight penalties based on feature distance intervals to reconstruct noise-resistant, high-dimensional physiological feature tensors.

Embodiment 38. The system of embodiment 37, wherein the sleep staging classification model comprises a deep learning network including sequentially cascaded layers of: deep residual feature extraction, a Temporal Convolutional Network (TCN), a bidirectional LSTM, and a multi-head attention mechanism, so as to output continuous sleep stage labels comprising at least one of: awake, light sleep, deep sleep, and REM states.

Embodiment 39. The system of any of embodiments 11-38, wherein the instructions operating on the at least one processor of the server cause the server to additionally include performing physiological metric quantification.

Embodiment 40. The system of embodiment 39, wherein physiological metric quantification comprises resting heart rate extraction and heart rate variability extraction.

Embodiment 41. The system of embodiment 40, wherein resting heart rate extraction comprises slicing filtered feature vectors using specific duration sliding windows sorted by standard deviation to extract stable intervals and selecting windows with largest average pulse intervals.

Embodiment 42. The system of embodiments 40 or 41, wherein heart rate variability extraction comprises calculating RMSSD in fixed windows after excluding pre-sleep and post-wake periods, while retaining intermediate intra-sleep awakenings, after removing outliers and excluding a predetermined percentage of anomalous data.

Embodiment 43. The system of embodiment 42, wherein the percentage is: between: 5-90%, 10-90%, 15-90%, 20-90%, 30-90%, 40-90%, 50-90%, 60-90%, 70-90%, 80-90%, or ranges therebetween, less than approximately: 90%, 80%, 70%, 60%, 50%, 40%, 30%, 20%, 10%, 5%, or less than 5% or approximately 5%, 10%, 20%, 30%, 40%, 50%, 60%, 70%, 80% or 90%.

Embodiment 44. The system of any of embodiments 1-43, wherein data from the PPG sensor is used by the system to analyze heart rate variability.

Embodiment 45. The system of embodiment 44, wherein the analysis of heart rate variability assesses autonomic nervous system balance and/or derives respiration rate from respiratory sinus arrhythmia during breathing cycles.

Embodiment 46. The system of any of embodiments 1-45, wherein the IMU sensor is configured to differentiate between micro-movements and/or to determine sleep position.

Embodiment 47. The system of embodiment 46, wherein the determined sleep position include any one or more of tossing and/or turning versus gross movements including one or more of waking up or getting out of bed.

Embodiment 48. The system of embodiments 46 or 47, wherein determined sleep position includes any one or more of back, side, and stomach positioning.

Embodiment 49. The system of any of embodiments 24-48, wherein closed-loop neuromodulation includes an internal cool-down period and/or a rolling limit mechanism.

Embodiment 50. The system of embodiment 49, wherein the inclusion of the internal cool-down period and/or the rolling limit mechanism is configured to form a safe, instantaneous, and adaptive edge-side intervention loop.

Embodiment 51. The system of any of embodiment 1-50, wherein the instructions operating on the at least one processor of the wearable device and/or a/the server cause the at least one processor of the wearable device and/or the server to use linear frequency match logic to identify when a user is slipping out of sleep and applies rhythmic stimulus to anchor the user by matching heart rate frequency in a range of In some embodiments, the frequency ranges from about 0.1 Hz to about 10 Hz, which may include one or another of subranges 0.1-9 Hz, 0.2-9 Hz, 0.3-9 Hz, 0.4-8 Hz, 0.5-8 Hz, 0.5-7 Hz, 0.5-6 Hz, 0.5-5 Hz, 0.5-4 Hz, 0.5-3 Hz, 0.5-2 Hz, and ranges therebetween.

Embodiment 52. The system of any of embodiments 1-51, wherein the instructions operating on the at least one processor of the wearable device and/or the server cause the at least one processor of the wearable device and/or the server to limit one or more intervention zones to micro-awakenings, wakes, and light-sleep states, and/or to establish veto zones so as to never intervene during an REM or deep sleep state of a user.

Embodiment 53. The system of any of embodiments 1-52, the wearable device further comprises mm Wave radar for non-contact sensing.

Embodiment 54. The system of any of embodiments 1-53, wherein instructions operating on one and/or another of one or more processors associated with the system include: HRV/heart rate mapping; Respiratory mapping; or HRV/heart rate mapping and respiratory mapping.

Embodiment 55. A method for sleep monitoring and intervention comprising: detecting at least one of heart rate signals and blood volume pulses of a user/individual; detecting at least one of micro-movements, gross movements, and postural position of the user; detecting wake-arousal of the user; and as a result of detecting wake-arousal, causing application of stimulation to at least one portion of the body of the user with one or more stimulus patterns so as to facilitate the user back to a sleeping state.

Embodiment 56. The method of embodiment 55, further comprising performing arousal scoring to detect wake-arousal of the user, and/or detecting is via a wearable device.

Embodiment 57. The method of embodiment 56, wherein one or more steps are performed via at least one processor of a/the wearable device (which may be used to, a bedside hub, and an external device and/or a server, each of which can comprise one or more processors.

Embodiment 58. The method of any of embodiments 55-57, further comprising communicating with a bedside hub.

Embodiment 59. The method of any of embodiments 55-57, further comprising performing at least one of: monitoring at least one of blue light exposure of the user and light pollution using an ambient light sensor, detecting acoustic events of the user including at least one of snoring, sleep apnea, and noise using a microphone; and monitoring climate conditions proximate an area of sleep using at least one of temperature and humidity sensors.

Embodiment 60. The method of embodiments 58 or 59, further comprising communicating between a bedside hub and a/the wearable device.

Embodiment 61. The method of any of embodiments 55-60, further comprising communicating between a/the wearable device, a/the bedside hub, and/or a server, with an external device using wireless communication means.

Embodiment 62. The method of any of embodiments 58-60, further comprising communicating between the wearable device and/or an external device using wireless communication means of a/the bedside hub.

Embodiment 63. The method of embodiments 61 or 62 wherein the external device includes a smartphone or other device configured for communication with a network.

Embodiment 64. The method of embodiment 63, wherein the network comprises the internet, the cloud, a LAN, and/or a WAN.

Embodiment 65. The method of any of embodiments 55-64, further comprising using a/the server to perform processing and analysis operations, and/or, such processing and analysis operations are performed on the wearable device, an external device, and/or a bedside hub.

Embodiment 66. The method of embodiment 65, further comprising instructions for causing at least one processor (e.g., of the server) to perform at least one of: a multimodal feature reconstruction using at least one of 1D convolutional kernels and Non-Maximum Suppression, and bio-analysis.

Embodiment 67. The method of embodiment 66, wherein multimodal feature reconstruction and/or bio-analysis is performed using deep learning networks.

Embodiment 68. The method of embodiment 67, wherein the use of deep-learning networks is with cascaded layers.

Embodiment 69. The method of any of embodiments 55-68, wherein a/the heart sensor, included with a/the wearable device, comprises a photoplethysmography (PPG) sensor.

Embodiment 70. The method of any of embodiments 55-69, wherein detecting utilizes an IMU provided for on a/the wearable device which comprises at least one accelerometer and/or at least one gyroscope.

Embodiment 71. The method of any of embodiments 55-69, wherein detecting utilizes an IMU provided for on a/the wearable device comprises one or more 3-axis accelerometers and one or more gyroscopes, and wherein detecting at least one of micro-movements, gross movements, and postural position uses the one or more 3-axis accelerometers and/or the one or more gyroscopes. Embodiment 72. The method of any of embodiments 55-71, wherein the stimulus patterns are rhythmic.

Embodiment 73. The method of any of embodiments 55-72, further comprising detecting use/wear of the wearable device on the user.

Embodiment 74. The method of embodiment 73, wherein the detecting use/wear of the wearable includes anti-spoofing wear detection.

Embodiment 75. The method of embodiment 74, wherein the anti-spoofing wear detection uses cascaded decision rules addressing at least one of: signal saturation, severe light leakage, wearer stillness, edge-side physical activity feature extraction.

Embodiment 76. The method of embodiment 75, wherein edge-side physical activity feature extraction includes asymmetric smoothing extraction.

Embodiment 77. The method of any of embodiments 55-76, wherein stimulation comprises neuromodulation.

Embodiment 78. The method of any of embodiments 55-76, wherein stimulation comprises closed-loop neuromodulation.

Embodiment 79. The method of any of embodiments 56-78, wherein arousal scoring occurs in real-time or substantially in real-time.

Embodiment 80. The method of any of embodiments 74-79, wherein anti-spoofing wear detection comprises at least one of: collecting high-frequency at least one of PPG and IMU signals of the user, down-sampling the at least one of PPG and IMU signals to a predetermined frequency, using sliding windows, extracting at least one of: a signal mean, a signal standard deviation, and one or more inter-window mean differences, and applying cascaded decision rules to intercept invalid data from non-wearing states, activating subsequent calculation(s) only when a valid wearing condition detected.

Embodiment 81. The method of any of embodiments 76-80, wherein asymmetric smoothing feature extraction performs frequency reduction and/or differential summation on high-frequency IMU signals.

Embodiment 82. The method of any of embodiments 76-81, wherein asymmetric smoothing includes fast-attack and slow-decay characteristics to fill stationary gaps so as to capture signs corresponding to wake-arousal with limited to no delay.

Embodiment 83. The method of any of embodiments 76-82, wherein asymmetric smoothing results in feature values to increase when body movement increases and decays slowly according to a preset forgetting factor when movement weakens, so as to effectively capture micro-movements in real-time.

Embodiment 84. The method of embodiment 83, wherein fragmented micro-movements of the user are captured in real-time.

Embodiment 85. The method of any of embodiments 78-84, wherein closed-loop neuromodulation comprises using an awake prediction model that inputs asymmetric smoothed features into a modified Oakley Arousal Scoring Model coupled with a weighted sliding window so as to predict light sleep or awake states significantly earlier than traditional algorithms.

Embodiment 86. The method of any of embodiments 78-85, further comprising evaluating continuous wake-up and high-frequency density of the user trigger conditions based on wake-arousal scores of the user; and dynamically calculating vibration duration based on current wake-arousal score without waiting for commands, wherein higher arousal scores correspond to deeper wakefulness states resulting in longer neurostimulation periods.

Embodiment 87. The method of any of embodiments 55-86, further comprising filtering data using at least one of the wearable device, a/the server, a/the bedside hub, and an external device. Embodiment 88. The method of embodiment 87, wherein filtering is via one or more filters to detect one or more peaks and/or one or more valleys in time, and/or compress high-frequency raw waveform data into low-volume sequences of peak/valley timestamps.

Embodiment 89. The method of embodiment 88, further comprising communicating a/the low-volume sequence(s) of peak/valley timestamps to a/the server.

Embodiment 90. The method of any of embodiments 55-86, further comprising applying a second-order cascaded Direct Form II Transposed high-pass and low-pass filter to PPG signals, detecting peaks and valleys in time domain, and/or compressing high-frequency raw waveform data into low-volume sequences of peak/valley timestamps for transmission to at least one of a/the wearable device, a/the bedside hub, an/the external device, and a/the server.

Embodiment 91. The method of any of embodiments 65-90, further comprising (which may be performed by one and/or another of a/the wearable device, a/the bedside hub, and/or an/the external device and/or a/the server) parsing received peak/valley sequences, calculating and/or merging time-sliding peak-to-peak interval distribution matrices, applying a one-dimensional convolutional kernel dual smoothing in distance and time domains, and applying Non-Maximum Suppression and dynamic weight penalties based on feature distance intervals to reconstruct noise-resistant, high-dimensional physiological feature tensors.

Embodiment 92. The method of embodiment 91, further comprising using classifying a sleep stage via a sleep staging classification model comprising at least one of: cascaded layers of deep residual feature extraction, Temporal Convolutional Network, bidirectional LSTM, and multi-head attention, so as to output continuous sleep stage labels comprising at least one of: awake, light sleep, deep sleep, and REM states.

Embodiment 93. The method of any of embodiments 65-92, further comprising causing the server to perform physiological metric quantification.

Embodiment 94. The method of embodiment 93, wherein physiological metric quantification comprises performing resting heart rate extraction and heart rate variability extraction.

Embodiment 95. The method of embodiment 94, wherein performing resting heart rate extraction comprises slicing filtered feature vectors using specific duration sliding windows sorted by standard deviation to extract most stable intervals and selecting windows with largest average pulse intervals.

Embodiment 96. The method of embodiments 94 or 95, wherein performing heart rate variability extraction comprises calculating RMSSD in fixed windows after excluding pre-sleep and post-wake periods, while retaining intermediate intra-sleep awakenings, after removing outliers and excluding a predetermined percentage of anomalous data.

Embodiment 97. The method of embodiment 96, wherein the percentage is: between: 5-90%, 10-90%, 15-90%, 20-90%, 30-90%, 40-90%, 50-90%, 60-90%, 70-90%, 80-90%, or ranges therebetween, less than approximately: 90%, 80%, 70%, 60%, 50%, 40%, 30%, 20%, 10%, 5%, or less than 5% or approximately 5%, 10%, 20%, 30%, 40%, 50%, 60%, 70%, 80% or 90%.

Embodiment 98. The method of any of embodiments 55-97, further comprising using data from the PPG sensor to analyze heart rate variability.

Embodiment 99. The method of embodiment 98, wherein the analysis of heart rate variability assesses autonomic nervous system balance and/or derives respiration rate from respiratory sinus arrhythmia during breathing cycles.

Embodiment 100. The method of any of embodiments 55-99, further comprising using a/the IMU sensor to differentiate between micro-movements and/or to determine sleep position.

Embodiment 101. The method of embodiment 100, wherein the determined sleep position include any one or more of tossing and/or turning versus gross movements including one or more of waking up or getting out of bed.

Embodiment 102. The method of embodiments 100 or 101, wherein determined sleep position includes any one or more of back, side, and stomach positioning.

Embodiment 103. The method of any of embodiments 78-102, wherein closed-loop neuromodulation includes using an internal cool-down period and/or a rolling limit mechanism.

Embodiment 104. The method of embodiment 103, wherein the inclusion of the internal cool-down period and/or the rolling limit mechanism forms a safe, instantaneous, and adaptive edge-side intervention loop.

Embodiment 105. The method of any of embodiments 55-104, further comprising using linear frequency match logic to identify when a user is slipping out of sleep and applying rhythmic stimulus to anchor the user by matching heart rate frequency in a predetermined frequency range which may include 0. 1-9 Hz, 0.2-9 Hz, 0.3-9 Hz, 0.4-8 Hz, 0.5-8 Hz, 0.5-7 Hz, 0.5-6 Hz, 0.5-5 Hz, 0.5-4 Hz, 0.5-3 Hz, 0.5-2 Hz, and ranges therebetween.

Embodiment 106. The method of any of embodiments 55-105, further comprising limiting one or more intervention zones to micro-awakenings, wakes, and light-sleep states, and/or establishing veto zones so as to never intervene during an REM or deep sleep state of a user.

Embodiment 107. The method of any of embodiments 55-106, performing non-contact sensing via mm Wave radar.

Embodiment 108. The method of any of embodiments 55-107, further comprising: HRV/heart rate mapping; Respiratory mapping; or HRV/heart rate mapping and respiratory mapping.

Embodiment 109. A sleep monitoring and intervention system comprising: a wearable device comprising a heart sensor to detect at least one of heart rate signals and blood volume pulses, an inertial measurement unit (IMU) sensor configured to detect at least one of micro-movements, gross movements, and postural position; a vibration motor configured to provide stimulation to at least one portion of the body with one or more stimulus patterns; and at least one processor including instructions operating thereon configured to cause the at least one processor to detect wake-arousal, and as a result thereof, cause the vibration motor to provide the stimulation so as to facilitate the user back to a sleeping state, a bedside hub configured to communicate with at least the wearable device and including at least one processor with instructions operating thereon; and a server and/or an external device comprising at least one processor with instructions operating thereon configured to cause the server to perform at least one of: a multimodal feature reconstruction using at least one of 1D convolutional kernels and Non-Maximum Suppression, and bio-analysis.

Embodiment 110. The system of embodiment 109, wherein the instructions operating on at least one of the processor of the wearable device, the bedside hub, and the server are configured to cause the processor to perform arousal scoring to detect wake-arousal.

Embodiment 111. The system of embodiments 109 or 110, wherein the at least one processor of any of the wearable device, the bedside hub and the server comprises a plurality of processors.

Embodiment 112. The system of any of embodiments 109-111, wherein the bedside hub includes at least one of: an ambient light sensor to monitor at least one of blue light exposure and light pollution, a microphone to detect acoustic events including at least one of snoring, sleep apnea, and noise, and at least one of temperature and humidity sensors to monitor climate conditions proximate an area of sleep.

Embodiment 113. The system of embodiments 109-112, wherein the external device includes a smartphone or other device configured for communication with a network.

Embodiment 114. A sleep monitoring and intervention wearable device comprising: a heart sensor to detect at least one of heart rate signals and blood volume pulses; an inertial measurement unit (IMU) sensor configured to detect at least one of micro-movements, gross movements, and postural position; a vibration motor configured to provide stimulation to at least one portion of the body with one or more stimulus patterns; and at least one processor including instructions operating thereon configured to cause the at least one processor to detect wake-arousal, and as a result thereof, cause the vibration motor to provide the stimulation so as to facilitate the user back to a sleeping state.

Embodiment 115. The device of embodiment 114, wherein the device communicates with at least one of: a bedside hub; and an external device and/or a remote server.

Embodiment 116. The device of any of embodiments 114-115, wherein the instructions are further configured to cause the at least one processor of the device to perform at least one of: a multimodal feature reconstruction using at least one of 1D convolutional kernels and Non-Maximum Suppression, and bio-analysis.

Embodiment 117. The device of any of embodiments 114-115, wherein the instructions are further configured to cause the at least one processor of at least one of the device, the bedside hub, the external device and the remote server to perform at least one of: a multimodal feature reconstruction using at least one of 1D convolutional kernels and Non-Maximum Suppression, and bio-analysis.

Embodiment 118. The device of embodiment 114, wherein the instructions operating on at least one of the processor of the wearable device cause the processor to perform arousal scoring to detect wake-arousal.

Embodiment 119. The device of embodiment 114, wherein the instructions operating on at least one of the processor of the wearable device, the bedside hub, the external device, cause the at least one processor to perform arousal scoring to detect wake-arousal.

Embodiment 120. The system of any of embodiments 114-119, wherein wearable device includes at least one of: an ambient light sensor to monitor at least one of blue light exposure and light pollution, a microphone to detect acoustic events including at least one of snoring, sleep apnea, and noise; and at least one of temperature and humidity sensors to monitor climate conditions proximate an area of sleep.

Embodiment 121. The system of any of embodiments 114-119, wherein at least one of the wearable device, the bedside hub, and the external device includes at least one of: an ambient light sensor to monitor at least one of blue light exposure and light pollution, a microphone to detect acoustic events including at least one of snoring, sleep apnea, and noise; and at least one of temperature and humidity sensors to monitor climate conditions proximate an area of sleep.

Embodiment 122. The system of embodiments 114-121, wherein the external device includes a smartphone or other device configured for communication with a network.

### Definitions & General Considerations

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

The indefinite articles “a” and “an,” as used herein in the specification and in the claims, unless clearly indicated to the contrary, should be understood to mean “at least one.” The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s). Additionally, “any and all” of certain recited items including a part(s), a structure(s), a function(s)/functionality, a clarification(s) or a step(s) (and the like) corresponds to certain embodiments only including one of such item (and in some embodiments, only such item), certain embodiments including two or more of such items (and in some embodiments, only two or more of such items), certain embodiments including substantially all of the items (and in some embodiments, only substantial number of the items), and certain embodiments including all of such items (and in some embodiment, only all of such embodiments).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined.

Other elements may optionally be present other than the elements specifically identified by the “and/or” clause, whether related or unrelated to those elements specifically identified. Thus, as a non-limiting example, a reference to “A and/or B”, when used in conjunction with open ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. The term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described or illustrated herein. More generally, those skilled in the art will readily appreciate that all structure, parameters, dimensions, materials, functionality, and configurations described herein are meant to be an example and that the actual structure, parameters, dimensions, materials, functionality, and configurations will depend upon the specific application or applications for which the inventive teachings is/are used. Those skilled in the art will recognize or be able to ascertain using no more than routine experimentation, many equivalents to the specific inventive embodiments described herein. It is therefore to be understood that the foregoing embodiments are presented by way of example only and that, within the scope of the claims supported by the present disclosure, and equivalents thereto, inventive embodiments may be practiced otherwise than as specifically described and claimed. Inventive embodiments of the present disclosure are also directed to each individual feature, system, article, structure, material, kit, functionality, step, and method described herein. In addition, any combination of two or more such features, systems, articles, structure, materials, kits, functionalities, steps, and methods, if such are not mutually inconsistent, is included within the inventive scope of the present disclosure. Moreover, some embodiments of this disclosure may be distinguishable from the prior art for specifically lacking one or more features/elements/functionality (i.e., claims directed to such embodiments can include negative limitations distinguishing said claim from the prior art).

Also, as noted, various inventive concepts may be embodied as one or more methods. The acts performed as part of a method may be ordered in any suitable way. Accordingly, embodiments may be constructed in which acts are performed in an order different than illustrated and described, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms. What is currently claimed:

1. A method for sleep monitoring and intervention comprising:
detecting at least one of heart rate signals and blood volume pulses of a user and/or detecting at least one of micro-movements, gross movements, and postural position of the user;
detecting or determining wake-arousal of the user; and
as a result of detecting or determining wake-arousal, applying stimulation to at least one portion of the body of the user with one or more stimulus patterns so as to facilitate the user back to a sleeping state.

2. The method of claim 1, further comprising performing arousal scoring to detect or determine wake-arousal of the user.

3. The method of claim 1, further comprising:
monitoring at least one of blue light exposure of the user and light pollution using an ambient light sensor proximate an area of sleep of the user,
detecting acoustic events proximate the area of sleep the user including at least one of snoring, sleep apnea, and noise using a microphone; and/or
monitoring climate conditions proximate the area of sleep using at least one of temperature and humidity sensors.

4. The method of claim 1, wherein detecting heart rate signals, blood volume pulses, and/or micro-movements, gross movements, and postural position of the user is via wearable device.

5. The method of claim 4, further comprising communicating with at least one of a bedside hub, an external device, and a server, using wireless communication means of the wearable device.

6. The method of claim 1, further comprising using a/the server to perform processing and analysis operations, or, such processing and analysis operations are performed on the wearable device, an external device, a bedside hub, or a combination of the foregoing.

7. The method of claim 6, further comprising performing at least one of: a multimodal feature reconstruction using at least one of 1D convolutional kernels and Non-Maximum Suppression, and bio-analysis.

8. The method of claim 7, wherein multimodal feature reconstruction and/or bio-analysis is performed using deep learning networks.

9. The method of claim 8, wherein the use of deep-learning networks is with cascaded layers.

10. The method of claim 1, wherein detecting utilizes an IMU which comprises one or more 3-axis accelerometers and one or more gyroscopes, and wherein detecting at least one of micro-movements, gross movements, and postural position uses the one or more 3-axis accelerometers and/or the one or more gyroscopes.

11. The method of claim 1, wherein the stimulus patterns are rhythmic.

12. The method of claim 4, further comprising detecting use/wear of the wearable device on the user, wherein detecting use/wear of the wearable includes anti-spoofing wear detection.

13. The method of claim 12, wherein the anti-spoofing wear detection uses cascaded decision rules addressing at least one of: signal saturation, severe light leakage, wearer stillness, edge-side physical activity feature extraction.

14. The method of claim 13, wherein edge-side physical activity feature extraction includes asymmetric smoothing extraction.

15. The method of claim 1, wherein stimulation comprises closed-loop neuromodulation.

16. The method of claim 4, wherein anti-spoofing wear detection comprises at least one of:
collecting high-frequency at least one of PPG and IMU signals of the user, down-sampling the at least one of PPG and IMU signals to a predetermined frequency,
using sliding windows, extracting at least one of: a signal mean, a signal standard deviation, and one or more inter-window mean differences,
and
applying cascaded decision rules to intercept invalid data from non-wearing states, activating subsequent calculation(s) only when a valid wearing condition detected.

17. The method of claim 14, wherein asymmetric smoothing feature extraction:
performs frequency reduction and/or differential summation on high-frequency IMU signals,
includes fast-attack and slow-decay characteristics to fill stationary gaps so as to capture signs corresponding to wake-arousal with limited to no delay, and/or.
results in feature values to increase when body movement increases and decays slowly according to a preset forgetting factor when movement weakens, so as to effectively capture micro-movements in real-time.

18. The method of claim 15, wherein closed-loop neuromodulation comprises using an awake prediction model that inputs asymmetric smoothed features into a modified Oakley Arousal Scoring Model coupled with a weighted sliding window so as to predict light sleep or awake states significantly earlier than traditional algorithms.

19. The method of claim 15, further comprising:
evaluating continuous wake-up and high-frequency density of the user trigger conditions based on wake-arousal scores of the user; and
dynamically calculating vibration duration based on current wake-arousal score without waiting for commands,
wherein higher arousal scores correspond to deeper wakefulness states resulting in longer neurostimulation periods.

20. The method of claim 1, further comprising filtering data using at least one of the wearable device, a/the server, a/the bedside hub, and an external device.
